(19) United States
(12) Patent Application Publication
(10) Pub. No.: US 2026/0015641 A1
(43) Pub. Date: Jan. 15, 2026

(54) METHODS FOR IN VITRO TRANSCRIPTION

(71) Applicant: ARCTURUS THERAPEUTICS, INC., San Diego, CA (US)

(72) Inventors: Brett Dallas HILL, San Diego, CA (US); Kyoung-Joo Jenny CHOI, Oceanside, CA (US)

(21) Appl. No.: 19/267,305

(22) Filed: Jul. 11, 2025

Related U.S. Application Data
(60) Provisional application No. 63/670,564, filed on Jul. 12, 2024.
(60) Provisional application No. 63/791,838, filed on Apr. 21, 2025.

(51) Int. Cl. C12P 19/34

(52) U.S. Cl. CPC C12P 19/34

(57) ABSTRACT

Disclosed herein are methods of producing transcribed RNA product with increased yield and reduced RNA degradation.

### CROSS-REFERENCES TO RELATED APPLICATIONS

This application claims the benefit of U.S. Provisional Application No. 63/670,564, filed Jul. 12, 2024, and U.S. Provisional Application No. 63/791,838, filed Apr. 21, 2025, both of which are incorporated herein by reference in their entirety and for all purposes.

### FIELD

The present application generally relates to methods for in vitro transcription.

### BACKGROUND

RNA-based therapeutics and vaccines are at the frontier of modem medicine providing hope for those suffering from genetic diseases and those fearing deadly pathogens. For example, messenger RNA (mRNA) can be used in protein replacement therapy to treat diseases caused by a lack of protein, or by defective proteins, such as in cystic fibrosis. If a gene has a mutation that stops it from producing protein or causes it to produce defective protein, mRNA medicine can provide a healthy version of the missing protein.

RNA-based vaccines have emerged as a new class of RNA medicines. RNA vaccines can be developed more rapidly than traditional vaccines in response to infectious disease outbreaks as shown by the first two vaccines to be licensed by the FDA for the prevention of COVID-19, a deadly viral infection caused by SARS-CoV-2.

One challenge in in vitro transcription (IVT) manufacturing that remains unaddressed is how to increase yield per given IVT volume and reduce RNA degradation. As RNA is generated, it begins to degrade, a factor which reduces RNA integrity (% full-length), leading to a lower quality product. Having higher percentages of non-full-length RNA would lead to reduction in efficacy as non-full-length RNAs would not translate the proteins of interest as efficiently. The goal is to increase RNA yield and reduce RNA degradation, thereby leading to improved RNA efficacy.

In manufacturing, there are many challenges associated with materials (e.g., costs, limited supplies), capacity conforming to good manufacturing practices (GMP) (e.g., limited slots available, significant time and labor required for preparation of a single run), and equipment (e.g., availability, validation, handling). Maximizing the amount of good quality mRNA manufactured at a given scale leads to reduction in material cost, number of expensive GMP runs, time, and labor, and even in some cases, eliminates the need of validating larger-scale equipment and processes. Thus, there is an urgent need to develop mRNA manufacturing processes that reliably and efficiently produce mRNA with increased yields and with reduced degradation.

### SUMMARY

As noted above, RNA-based therapeutics and vaccines are at the frontier of modem medicine providing hope for those suffering from a variety of diseases and illnesses. Challenges remain for producing the best possible RNA-based therapeutics. The present disclosure relates to various surprising and unexpected innovations in the production of RNA based therapies and methods related the same.

The disclosure also relates to the discovery that adding a chelating compound to the IVT can improve RNA integrity, thus increasing RNA quality, shelf life, and ultimately efficacy. Magnesium is an essential cofactor for the synthesis of RNA by T7 RNA polymerase. However, magnesium also catalyzes RNA hydrolysis which causes RNA strand breakage during the IVT reaction, reducing the percentage of full-length RNA that is produced. The concentration of free magnesium is believed to increase throughout the IVT reaction, further exacerbating RNA degradation. Addition of specific chelators to the IVT can improve the RNA integrity, presumably by chelating the free magnesium over the course of the reaction and reducing the rate of RNA hydrolysis. RNA integrity can be improved by 10% using this approach, allowing one to achieve nearly 90% RNA integrity for very long RNAs (˜12 kb), which is very challenging using the current state of the art.

In one aspect, the present disclosure is drawn to a method for producing a single-stranded RNA product, the method comprising a) reacting a transcription reaction mixture comprising a buffer solution comprising Mg²⁺, a DNA template, ribonucleoside tri-phosphates (rNTPs), and RNA polymerase; wherein the rNTPs comprise adenine (A), cytosine (C), guanine (G), and uracil (U) nucleosides, or analogs thereof, at a molar ratio that is within 5-10% of the ratio of A:C:G:T in the DNA template; b) controlling the rate of the transcription reaction of step (a) to be about 10 mM rNTP/hour to about 25 mM rNTP/hour; and c) stopping the transcription reaction by digesting the DNA template with deoxyribonuclease (DNase) or quenching the RNA polymerase with a chelator; thereby producing the single-stranded RNA product.

In some embodiments, the chelator is selected from NTA (nitrilotriacetic acid), EDTA (ethylenediaminetetraacetic acid), EDTPO (ethylenediamine tetra(methylene phosphoric acid)), EGTA (ethylene glycol-bisO-aminoethyl ether)-N,N,N′,N′-tetraacetic acid, BAPTA (1,2-Bis(2-aminophenoxy)ethane-N,N,N′,N′-tetraacetic acid), DFOA (Deferoxamine Mesylate), Dimethoxynitrophenamine (1-(2-Nitro-4,5-dimethoxyphenyl)-1,2-diaminoethane-N,N,N′,N′-tetraacetic Acid)), CDTA (1,2-cyclohexylenedinitrilo)tetraacetic acid), DPTA (diethylenetriaminepentaacetic acid), PIH (pyridoxal isonicotinoyl hydrazone), malonic acid, and TPEN (N′-Tetrakis(2-pyridylmethyl)ethylenediamine).

In some embodiments, the DNA template is in a solution comprising 0 mM to 1200 mM NaCl to produce a salt-spiked DNA template prior to introduction to the transcription reaction mixture.

In some embodiments, the NaCl is present in the transcription reaction mixture at a concentration of between about 0 mM and about 85 mM.

In some embodiments, the NaCl is present in the transcription reaction mixture at a concentration of between about 0 mM and about 70 mM.

In some embodiments, the NaCl is present in the transcription reaction mixture at a concentration of between about 0 mM and about 65 mM.

In some embodiments, the concentration of rNTPs ranges from about 25 mM to about 55 mM.

In some embodiments, the concentration of RNA polymerase is about 50 units/μL to about 400 units/μL.

In some embodiments, the concentration of RNA polymerase is about 50 units/μL to about 200 units/μL.

In some embodiments, the concentration of RNA polymerase is about 50 units/μL to about 150 units/μL.

In some embodiments, the method produces about 10 to about 20 g/L of single-stranded RNA product. In some embodiments the method includes collecting or obtaining about 10 to about 20 g/L of single-stranded RNA product.

In some embodiments, the single-stranded RNA product is about 90% to about 100% pure. In some embodiments the method includes collecting or obtaining a single-stranded RNA product that is about 90% to about 100% pure.

In some embodiments, the single-stranded RNA product is about 75% to about 100% pure. In some embodiments the method includes collecting or obtaining a single-stranded RNA product that is about 75% to about 100% pure.

In some embodiments, the transcription reaction is stopped after about 60 minutes to about 240 minutes.

In some embodiments, the transcription reaction is stopped after about 120 minutes to about 180 minutes.

In some embodiments, the DNA template is linearized.

In some embodiments, an RNA polymerase KU activity per mL of transcription reaction mixture is greater than or equal to 125 KU RNA polymerase activity.

In some embodiments, the reaction mixture of step (a) further comprises RNase inhibitor, inorganic pyrophosphatase, or both.

In some embodiments, the initial pH of the reaction mixture is in a range from about 6.5 to about 8.0.

In some embodiments, step (a) and step (b) are each independently conducted at a temperature in a range from about 30° C. to about 40° C.

In some embodiments, the RNA polymerase is selected from T7 polymerase, Escherichia coli (E. Coli) polymerase, SP6 polymerase, T3 polymerase, or mutants thereof.

In some embodiments, the transcription reaction mixture of step (a) further comprises an initiating RNA capping reagent.

In some embodiments, the transcription reaction mixture of step (a) further comprises one or more solvents selected from the group consisting of ethanol in a concentration of about 1 to about 10% v/v, isopropyl alcohol in a concentration of about 1 to about 10% v/v, methanol in a concentration of about 1 to about 10% v/v, and acetonitrile in a concentration of about 1 to about 8% v/v.

In some embodiments, the total concentration of the one or more solvents does not exceed 10% v/v of the transcription reaction mixture of step (a).

In some embodiments, the rate of reaction is controlled by having the initial total rNTP concentration be in the range of about 20 mM to about 60 mM.

In some embodiments, the rate of reaction is controlled by having the initial total rNTP concentration be in the range of about 20 mM to about 60 mM and the Mg²⁺ concentration be in the range of about 15 mM to about 80 mM.

In some embodiments, the ratio of adenine nucleosides, guanine nucleosides, cytosine nucleosides, and uracil nucleosides, or analogs thereof, are tuned to be within 5% of the ratio of adenine, guanine, cytosine, and thymine bases on the linear DNA template.

In some embodiments, a yield of RNA transcript is increased by at least 5%, at least 10%, at least 15%, at least 20%, or at least 25% as compared to a transcription reaction in which the ratio of adenine nucleosides, guanine nucleosides, cytosine nucleosides, and uracil nucleosides, or analogs thereof, has not been tuned to be within 5% of the ratio of adenine, guanine, cytosine, and thymine bases on the linear DNA template. In some embodiments, the methods including obtaining or collecting a yield of RNA transcript that is at least 5%, at least 10%, at least 15%, at least 20%, or at least 25% as compared to a transcription reaction in which the ratio of adenine nucleosides, guanine nucleosides, cytosine nucleosides and uracil nucleosides, or analogs thereof, has not been tuned to be with 5% of the ratio of adenine, guanine, cytosine, and thymine bases on the linear DNA template.

In some embodiments, the rNTPs comprise adenine (A), cytosine (C), guanine (G), and uracil (U) nucleosides, or analogs thereof, at a molar ratio of about 10:13:13:6 (A:C:G:U).

In some embodiments, the rate of the transcription reaction of step (a) is controlled to be about 13 mM rNTP/hour to about 20 mM rNTP/hour.

In some embodiments, the rate of transcription is controlled by increasing the concentration of rNTPs and/or decreasing the concentration of RNA polymerase.

In some embodiments, the rate of transcription is controlled by changing the temperature, salt concentration, magnesium concentration, pH, or a combination thereof.

In some embodiments, the chelator can include one or more of NTA or EDTPO.

In some embodiments, the concentration of NTA is about 50 mM.

In some embodiments, the RNA transcript is at least 4000 nucleotides in length.

In some embodiments, the method further comprises adding ammonium hydroxide to the NTA, for example, prior to reacting the NTA with the transcription reaction mixture.

In some embodiments, the concentration of ammonium hydroxide is about 25 mM.

In some embodiments, the method further comprises adding additional chelator to the reaction as the reaction proceeds.

In another aspect, provided herein is a method for producing a single-stranded RNA product, the method comprising a) reacting a transcription reaction mixture comprising a buffer solution comprising Mg²⁺, a DNA template, ribonucleoside tri-phosphates (rNTPs), and RNA polymerase; wherein the rNTPs comprise adenine (A), cytosine (C), guanine (G), and uracil (U) nucleosides, or analogs thereof, at a molar ratio that is within 5-10% of the ratio of A:C:G:T in the DNA template, and wherein the transcription reaction results in the depletion of rNTPS, and b) stopping the transcription reaction prior to a point in which the rNTPs have been depleted to between about 0% to 35% of the original amount of rNTPs in the reaction mixture; thereby producing the single-stranded RNA product.

In some embodiments, the purity of the RNA transcript product as determined by measuring the percentage of full-length RNA in the RNA transcript product is increased as compared to an in vitro transcription wherein the transcription reaction was not stopped when the rNTPs have been depleted to about 0% to about 35% of the original amount of rNTPs in the reaction mixture. In some embodiments, the methods can include determining the purity of the RNA transcript.

In some embodiments, the reaction is stopped by digesting the DNA template with deoxyribonuclease (DNase) or quenching the RNA polymerase with a chelator; thereby producing the single-stranded RNA product.

In some embodiments, the chelator for stopping the IVT reaction is selected from one or more of NTA (nitrilotriacetic acid), EDTA (ethylenediaminetetraacetic acid), EDTPO (ethylenediamine tetra(methylene phosphoric acid)), EGTA (ethylene glycol-bisO-aminoethyl ether)-N,N,N′,N′-tetraacetic acid, BAPTA (1,2-Bis(2-aminophenoxy)ethane-N,N,N′,N′-tetraacetic acid), DFOA (Deferoxamine Mesylate), Dimethoxynitrophenamine (1-(2-Nitro-4,5-dimethoxyphenyl)-1,2-diaminoethane-N,N,N′,N′-tetraacetic Acid)), CDTA (1,2-cyclohexylenedinitrilo)tetraacetic acid), DPTA (diethylenetriaminepentaacetic acid), PIH (pyridoxal isonicotinoyl hydrazone), malonic acid, and TPEN (N′-Tetrakis(2-pyridylmethyl)ethylenediamine).

In some embodiments, the DNA template is in a solution comprising 0 mM to 1200 mM NaCl to produce a salt-spiked DNA template prior to introduction to the transcription reaction mixture.

In some embodiments, the NaCl is present in the transcription reaction mixture at a concentration of between about 0 mM and about 85 mM.

In some embodiments, the concentration of rNTPs ranges from about 25 mM to about 55 mM.

In some embodiments, the concentration of RNA polymerase is about 50 units/μL to about 400 units/μL.

In some embodiments, the method produces about 10 to about 20 g/L of single-stranded RNA product. In some embodiments, the methods can include obtaining or collection about 10 to about 20 g/L of single-stranded RNA product.

In some embodiments, the single-stranded RNA product is about 75% to about 100% pure. In some embodiments the method includes collecting or obtaining a single-stranded RNA product that is about 75% to about 100% pure

In some embodiments, the transcription reaction is stopped after about 60 minutes to about 240 minutes.

In some embodiments, the transcription reaction is stopped after about 120 minutes to about 180 minutes.

In some embodiments, the DNA template is linearized.

In some embodiments, the reaction mixture of step (a) further comprises RNase inhibitor, inorganic pyrophosphatase, or both.

In some embodiments, the initial pH of the reaction mixture is in a range from about 6.5 to about 8.0.

In some embodiments, the RNA polymerase is selected from T7 polymerase, Escherichia coli (E. Coli) polymerase, SP6 polymerase, T3 polymerase, or mutants thereof.

In some embodiments, the transcription reaction mixture of step (a) further comprises an initiating RNA capping reagent.

In some embodiments, the transcription reaction mixture of step (a) further comprises one or more solvents selected from the group consisting of ethanol in a concentration of about 1 to about 10% v/v, isopropyl alcohol in a concentration of about 1 to about 10% v/v, methanol in a concentration of about 1 to about 10% v/v, and acetonitrile in a concentration of about 1 to about 8% v/v.

In some embodiments, the total concentration of the one or more solvents does not exceed 10% v/v of the transcription reaction mixture of step (a).

In some embodiments, the ratio of adenine nucleosides, guanine nucleosides, cytosine nucleosides, and uracil nucleosides are tuned to be within 5% of the ratio of adenine, guanine, cytosine, and thymine bases on the linear DNA template.

In some embodiments, a yield of RNA transcript is increased by at least 5%, at least 10%, at least 15%, at least 20%, or at least 25% as compared to a transcription reaction in which the ratio of adenine nucleosides, guanine nucleosides, cytosine nucleosides, and uracil nucleosides has not been tuned to be within 5% of the ratio of adenine, guanine, cytosine, and thymine bases on the linear DNA template. In some embodiments, the methods including obtaining or collecting a yield of RNA transcript that is at least 5%, at least 10%, at least 15%, at least 20%, or at least 25% as compared to a transcription reaction in which the ratio of adenine nucleosides, guanine nucleosides, cytosine nucleosides and uracil nucleosides, or analogs thereof, has not been tuned to be with 5% of the ratio of adenine, guanine, cytosine, and thymine bases on the linear DNA template.

In some embodiments, the rNTPs comprise adenine (A), cytosine (C), guanine (G), and uracil (U) nucleosides, or analogs thereof, at a molar ratio of about 10:13:13:6 (A:C:G:U).

In yet another aspect, the present disclosure provides a method for producing a single-stranded RNA product, the method comprising a) reacting a transcription reaction mixture comprising a buffer solution comprising Mg²⁺, a DNA template, ribonucleoside tri-phosphates (rNTPs), and RNA polymerase; wherein the rNTPs comprise adenine (A), cytosine (C), guanine (G), and uracil (U) nucleosides, or analogs thereof, at a molar ratio that is within 5-10% of the ratio of A:C:G:T in the DNA template; and b) controlling the rate of the transcription reaction of step (a) to be about 10 mM rNTP/hour to about 25 mM rNTP/hour, thereby producing the single-stranded RNA product.

In some embodiments, the DNA template is in a solution comprising 50 mM to 1200 mM NaCl to produce a salt-spiked DNA template prior to introduction to the transcription reaction mixture.

In some embodiments, the NaCl is present in the transcription reaction mixture at a concentration of between about 60 mM and about 85 mM.

In some embodiments, the concentration of rNTPs ranges from about 25 mM to about 55 mM.

In some embodiments, the concentration of RNA polymerase is about 50 units/μL to about 400 units/μL.

In some embodiments, the method produces about 14 to about 20 g/L of single-stranded RNA product.

In some embodiments, the single-stranded RNA product is about 75% to about 100% pure.

In some embodiments, the DNA template is linearized.

In some embodiments, the reaction mixture of step (a) further comprises RNase inhibitor, inorganic pyrophosphatase, or both.

In some embodiments, the initial pH of the reaction mixture is in a range from about 6.5 to about 8.0.

In some embodiments, step (a) and step (b) are each independently conducted at a temperature in a range from about 30° C. to about 40° C.

In some embodiments, the RNA polymerase is selected from T7 polymerase, Escherichia coli (E. Coli) polymerase, SP6 polymerase, T3 polymerase, or mutants thereof.

In some embodiments, the transcription reaction mixture of step (a) further comprises an initiating RNA capping reagent.

In some embodiments, the transcription reaction mixture of step (a) further comprises one or more solvents selected from the group consisting of ethanol in a concentration of about 1 to about 10% v/v, isopropyl alcohol in a concentration of about 1 to about 10% v/v, methanol in a concentration of about 1 to about 10% v/v, and acetonitrile in a concentration of about 1 to about 8% v/v.

In some embodiments, the total concentration of the one or more solvents does not exceed 10% v/v of the transcription reaction mixture of step (a).

In some embodiments, the rate of reaction is controlled by having the initial total rNTP concentration be in the range of about 20 mM to about 60 mM.

In some embodiments, the rate of reaction is controlled by having the initial total rNTP concentration be in the range of about 20 mM to about 60 mM and the Mg²⁺ concentration be in the range of about 15 mM to about 80 mM.

In some embodiments, the ratio of adenine nucleosides, guanine nucleosides, cytosine nucleosides, and uracil nucleosides are tuned to be within 5% of the ratio of adenine, guanine, cytosine, and uracil bases on the linear DNA template.

In some embodiments, a yield of RNA transcript is increased by at least 5%, at least 10%, at least 15%, at least 20%, or at least 25% as compared to a transcription reaction in which the ratio of adenine nucleosides, guanine nucleosides, cytosine nucleosides, and uracil nucleosides has not been tuned to be within 5% of the ratio of adenine, guanine, cytosine, and thymine bases on the linear DNA template. In some embodiments, the methods including obtaining or collecting a yield of RNA transcript that is at least 5%, at least 10%, at least 15%, at least 20%, or at least 25% as compared to a transcription reaction in which the ratio of adenine nucleosides, guanine nucleosides, cytosine nucleosides and uracil nucleosides, or analogs thereof, has not been tuned to be with 5% of the ratio of adenine, guanine, cytosine, and thymine bases on the linear DNA template.

In some embodiments, the rNTPs comprise adenine (A), cytosine (C), guanine (G), and uracil (U) nucleosides, or analogs thereof, at a molar ratio of about 10:13:13:6 (A:C:G:U).

In some embodiments, the rate of the transcription reaction of step (a) is controlled to be about 13 mM rNTP/hour to about 20 mM rNTP/hour.

In some embodiments, the rate of transcription is controlled by increasing the concentration of rNTPs and/or decreasing the concentration of RNA polymerase.

In some embodiments, the rate of transcription is controlled by changing the temperature, salt concentration, magnesium concentration, pH, or a combination thereof.

In yet another aspect, the present disclosure provides a method for producing a single-stranded RNA product, the method comprising a) reacting a transcription reaction mixture comprising a buffer solution comprising Mg²⁺, a DNA template, ribonucleoside tri-phosphates (rNTPs), and RNA polymerase; wherein the rNTPs comprise adenine (A), cytosine (C), guanine (G), and uracil (U) nucleosides, or analogs thereof, at a molar ratio that is within 5-10% of the ratio of A:C:G:T in the DNA template; b) controlling the rate of the transcription reaction of step (a) to be about 10 mM rNTP/hour to about 25 mM rNTP/hour; and c) stopping the transcription reaction prior to a point in which the rNTPs have been depleted to between about 0% to 35% of the original amount of rNTPs in the reaction mixture; thereby producing the single-stranded RNA product.

In yet another aspect, the present disclosure provides a composition for in vitro transcription of a nucleic acid, the composition comprising a) ribonucleotide triphosphates (rNTPs), wherein the rNTPs include or consist of adenine (A), cytosine (C), guanine (G), and uracil (U) at a molar ratio of 10:13:13:6 (A:C:G:U); b) Mg²⁺; c) a DNA template; and d) RNA polymerase. In some aspects, the composition can include one or more additional excipients, for example, one ore more excipients associated with in vitro transcription. In some embodiments, the composition is in vitro.

In yet another aspect, the present disclosure provides a kit for in vitro transcription of a nucleic acid, the kit comprising a) ribonucleotide triphosphates (rNTPs), wherein the rNTPs consist of adenine (A), cytosine (C), guanine (G), and uracil (U) at a molar ratio of 10:13:13:6 (A:C:G:U); b) Mg²⁺; c) a DNA template; and d) RNA polymerase.

In yet another aspect, the present disclosure provides a method for reducing the degradation of an RNA transcript having a size larger than 4000 nucleotides, comprising reacting a transcription reaction mixture comprising a buffer solution comprising Mg²⁺, a DNA template, ribonucleoside tri-phosphates (rNTPs), and RNA polymerase; wherein the rNTPs comprise adenine (A), cytosine (C), guanine (G), and uracil (U) nucleosides, or analogs thereof; with a chelator.

In some embodiments, the chelator can be one or more of NTA (nitrilotriacetic acid), EDTA (ethylenediaminetetraacetic acid), EDTPO (ethylenediamine tetra(methylene phosphoric acid)), EGTA (ethylene glycol-bisO-aminoethyl ether)-N,N,N′,N′-tetraacetic acid, BAPTA (1,2-Bis(2-aminophenoxy)ethane-N,N,N′,N′-tetraacetic acid), DFOA (Deferoxamine Mesylate), Dimethoxynitrophenamine (1-(2-Nitro-4,5-dimethoxyphenyl)-1,2-diaminoethane-N,N,N′,N′-tetraacetic Acid)), CDTA (1,2-cyclohexylenedinitrilo)tetraacetic acid), DPTA (diethylenetriaminepentaacetic acid), PIH (pyridoxal isonicotinoyl hydrazone), and TPEN (N′-Tetrakis(2-pyridylmethyl)ethylenediamine).

In some embodiments, the chelator comprises EDTPO.

In some embodiments, the chelator comprises NTA.

In some embodiments, the chelator comprises EDTPO or EDTA at a concentration ranging from about 5 mM to about 50 mM.

In some embodiments, the concentration of EDTPO or EDTA is about 10 mM.

In some embodiments, the chelator comprises NTA or EGTA at a concentration ranging from about 1 mM to about 120 mM.

In some embodiments, the concentration of the NTA or EGTA is about 50 mM.

In some embodiments, the concentration of the NTA is about 50 mM.

In some embodiments, the RNA transcript is at least 4000 nucleotides in length.

In some embodiments, the method further comprises adding ammonium hydroxide, sodium hydroxide, or Tris base to the NTA, for example, prior to reacting the NTA with the transcription reaction mixture.

In some embodiments, the method comprises adding ammonium hydroxide at a concentration ranging from about 20 mM to about 60 mM.

In some embodiments, the method comprises adding ammonium hydroxide at a concentration of about 25 mM.

In some embodiments, the method comprises adding ammonium hydroxide at a concentration of about 50 mM.

In some embodiments, the method comprises adding sodium hydroxide at a concentration ranging from about 20 mM to about 60 mM.

In some embodiments, the method comprises adding sodium hydroxide at a concentration of about 25 mM.

In some embodiments, the method comprises adding sodium hydroxide at a concentration of about 50 mM.

In some embodiments, the method comprises adding Tris base at a concentration ranging from about 20 mM to about 60 mM.

In some embodiments, the method comprises adding Tris base at a concentration of about 25 mM.

In some embodiments, the method comprises adding Tris base at a concentration of about 50 mM.

In some embodiments, the method further comprises adding additional chelator to the reaction as the reaction proceeds.

In some embodiments, the chelator comprises a non-carboxylic acid chelator.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1 is a graph showing concentrations of the unreacted NTPs and completely transcribed RNA over the course of an in vitro transcription (IVT). Samples of IVT reaction stopped at the indicated timepoints (in minutes) were isolated and run on PrimaS multi-modal purification columns to separate unreacted NTPs and complete RNA. Note that GTP depletes before other NTPs around 90 minutes, indicating that the ratio of NTPs needs adjustment.

FIGS. 2A and 2B are graphs showing yield and % RNA integrity of mRNA.

FIG. 2A is a line graph showing the yield of mRNA with different ratios and amounts of NTPs from IVT reactions stopped at the indicated timepoints. The top two lines show yield under conditions that best match the NTP stoichiometry of the mRNA, whereas the middle line shows the standard NTP ratio. FIG. 2B is a bar graph showing the integrity of mRNA with different NTP ratios. An NTP ratio of 12:15:15:7 (ATP:CTP:GTP:UTP concentration in millimolar) leads to a similar level of mRNA integrity as the standard ratio of 12:12:12:6 (A:C:G:U).

FIG. 3 is a diagram showing the harvest time window for both improved IVT conditions and standard IVT conditions. The improved IVT reaction has a larger window to stop the reaction while still obtaining high yield and purity compared to the standard conditions. Without being bound by theory, it is believed the extra NTPs and lower T7 RNA polymerase concentration slow down the reaction while increasing the yield.

FIG. 4 shows that RNAs with longer length have increased rate of degradation of % full length transcript (“RNA Integrity”) than shorter RNAs.

FIG. 5 is an overlay of electropherograms from capillary electrophoresis of RNA produced in IVT reactions with different chelators. The shoulder on the left-hand side of the peak is RNA that is not full length. This shoulder is reduced with the addition of chelators.

FIGS. 6A-6C show the relationship between yield, RNA integrity, and turbidity (A500). FIG. 6A shows data upon addition of citrate chelator. FIG. 6B shows data upon addition of EDTA chelator. FIG. 6C shows data is upon addition of NTA chelator. The balance of magnesium acetate (MgOAc) and chelator concentrations are important to achieve both high RNA integrity and yield.

FIG. 7 shows the relationship between yield and turbidity (A500) for the data in FIGS. 6A-6C. Overall, chelators do not reduce turbidity significantly compared to the control reaction having no chelator addition.

FIG. 8 shows the effect of chelators in the IVT on the efficiency of the DNAse I digestion. The DNAse I reaction was performed immediately after IVT without any prior purification. Residual plasmid DNA (pDNA) was measured using qPCR with sequence specific primer.

### DETAILED DESCRIPTION

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, figures and detailed description are to be regarded as illustrative in nature and not as restrictive.

### Definitions

Unless defined otherwise, all technical and scientific terms used herein have the same meaning as commonly understood by one of ordinary skill in the art to which this disclosure belongs. In this specification and in the claims that follow, reference will be made to a number of terms that shall be defined to have the following meanings:

The terminology used herein is for the purpose of describing particular embodiments only and is not intended to be limiting. As used herein, the singular forms “a”, “an” and “the” are intended to include the plural forms as well, unless the context clearly indicates otherwise.

“Optional” or “optionally” means that the subsequently described event or circumstance can or cannot occur, and that the description includes instances where the event or circumstance occurs and instances where it does not.

The term “about,” when used before a numerical designation, e.g., temperature, time, amount, concentration, and such other, including a range, indicates approximations which may vary by (+) or (−) 10%, 5%, 1%, or any subrange or subvalue there between. Preferably, the term “about” when used with regard to an amount means that the amount may vary by +/−10%.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this disclosure.

The term “isolated,” “isolating,” “purified,” and the like, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high-performance liquid chromatography. A nucleic acid that is the predominant species present in a preparation is substantially purified.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non-limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g., polynucleotides contemplated herein include any types of RNA, e.g., mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double-strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double-bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press; which is incorporated herein by reference in its entirety for all of its teachings and disclosure, including all composition, materials and methodologies) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds (each of which is incorporated herein by reference in its entirety for all of its teachings and disclosure, including all composition, materials and methodologies. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

As used herein, the term “expression” is used in accordance with its plain ordinary meaning and refers to any step involved in the production of a polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “transcription” as used herein, generally refers to the process of copying a segment of DNA into RNA, where the segments of DNA transcribed into RNA molecules that encode proteins produce mRNA. In embodiments, the segments of DNA that are copied into RNA molecules are referred to as non-coding RNAs.

The term “polymerase” as used herein, generally refers to an enzyme that catalyzes the synthesis of DNA or RNA whose sequence is complementary to the original template. In embodiments, RNA polymerase is the enzyme that synthesizes RNA from a DNA template. Polymerases that are suitable for the compositions and methods described herein are described further below.

The term “template” as used herein, generally refers to the antisense DNA strand. In embodiments, the cell uses the antisense strand as a template for producing messenger RNA (mRNA) that directs the synthesis of a protein. In embodiments, the term “linear DNA (L.DNA) template” generally refers to DNA antisense strands that have been uncoiled or linearized by the use of restriction enzyme or are PCR amplicons.

The term “DNase” as used herein, generally refers to deoxyribonuclease, which is an enzyme that catalyzes the hydrolytic cleavage of phosphodiester bonds that connect/link nucleotides in the DNA backbone.

As used herein, “transcribed RNA product,” “in vitro transcribed RNA,” “in vitro-synthesized RNA” and the like generally refers to mRNA that is synthesized or prepared using a method comprising in vitro transcription of one or more DNA templates by an RNA polymerase. In embodiments, the in vitro-synthesized RNA encodes (or exhibits a coding sequence of) at least one protein or polypeptide. In some embodiments, the RNA encodes at least one protein that is capable of effecting a biological or biochemical effect when repeatedly or continuously introduced into a human or animal cell (e.g., a mammalian cell). In some embodiments, the disclosure comprises an RNA composition comprising or consisting of in vitro-synthesized RNA that encodes one protein or polypeptide. In embodiments, the disclosure comprises an RNA composition comprising or consisting of a mixture of multiple different in vitro-synthesized ssRNAs or mRNAs, each of which encodes a different protein. Other embodiments of the disclosure comprise an RNA composition comprising or consisting of in vitro-synthesized ssRNA that does not encode a protein or polypeptide, but instead exhibits the sequence of at least one long non-coding RNA (ncRNA). Still other embodiments comprise various reaction mixtures, kits and methods that comprise or use an RNA composition.

The terms “substantially free of dsRNA,” “virtually free of dsRNA,” “essentially free of dsRNA,” “practically free of dsRNA,” “extremely free of dsRNA,” or “absolutely free of dsRNA,” as used herein, respectively, that less than about: 0.5%, 0.1%, 0.05%, 0.01%, 0.001% or 0.0002% of the mass of the RNA in the treated ssRNA composition is dsRNA of a size greater than about 40 base pairs.

In embodiments, the one or more in vitro transcribed RNAs are substantially free of uncapped RNAs that exhibit a 5′-triphosphate group (which are considered to be one type of “contaminant RNA molecules” herein). As used herein, the RNA product is at least 50%, 60,%, 70%, 80%, 90%, 91%, 92%, 93%, 94%, 95%, 96%, 97%, 98%, 99%, 99.5%, or 99.99% free of uncapped RNAs that exhibit a 5′-triphosphate group.

As used herein, the term “salt spiking” refers to the addition of sodium chloride (NaCl) or an equivalent salt to the L.DNA prior to the addition of L.DNA to the in vitro transcription vessel (IVT).

As used herein, the term “quenching” refers to a process of deactivating any unreacted reagents.

As used herein, the term “ribonucleoside tri-phosphate” or “rNTP” refers to a nucleoside containing a nitrogenous base bound to ribose, with three phosphate groups bound to the ribose. They are the molecular precursors of RNA, which is a chain of nucleotides made through the process of transcription. rNTPs can include natural or modified nucleosides, i.e., analogs thereof.

As used herein, the term “single-stranded RNA” or “single-stranded nucleic acid” refers to an RNA molecule or a nucleic acid molecule that comprises only one strand, as opposed to an RNA molecule or a nucleic acid molecule that comprises two strands (i.e., is “double-stranded”).

As used herein, the term “pure,” “purity,” or “% pure” refers to a single-stranded RNA product that does not contain, or contains only a percentage of certain components (e.g., contaminants like cellular proteins or other undesired nucleic acid species). For example, a single-stranded RNA product that is 90% pure has 90% of the desired nucleic acid and 10% contaminants. A pure product may be free of undesired components (such as double-stranded RNA). Purity may also refer to what percentage of an RNA molecule is full length, e.g., in a product with 90% purity, 90% of the RNA molecules are full length.

As used herein, the term “reducing degradation” refers to improving the integrity of RNA transcripts, by increasing the percentage of full-length products generated.

As used herein, the term “Unit,” “Units,” or “U” in the context of “units/μL” refers to a standardized measure of enzyme activity, such as the amount of RNA polymerase added to a transcription reaction. The specific definition can vary slightly between manufacturers, but generally refers to the amount of RNA polymerase enzyme needed to incorporate a specific amount of substrate (e.g., ATP) into an acid-insoluble product under defined conditions (temperature, time).

Described herein are methods of producing transcribed RNA product with increased yield and reduced RNA degradation.

### Methods

Applicant discovered that by slowing down the kinetics of in vitro transcription, the RNA is produced more slowly, allowing for a large window in which to stop the reaction. This leads to improved RNA yield and integrity. To slow down the kinetics, Applicant has found that one can increase the amount of nucleoside triphosphates (NTPs) in the reaction, as well as increasing the concentrations of salt and the polymerase enzyme. Applicant has also discovered that stopping the IVT reaction at a point prior to depleting the NTPs, results in increased RNA quality, specifically the purity (% full-length), of the in vitro transcribed mRNA. The temporal window where the IVT can be stopped before depleting the NTPs has also been widened by slowing down the IVT reaction through increasing the NTP and Mg²⁺ concentrations. Finally, the ratio of individual NTPs can be and were tuned to increase the achievable mRNA yield by 25%, for example, while only increasing the total NTPs by 15%.

In some embodiments, the present disclosure is drawn to a method for producing a single-stranded RNA product, the method comprising a) reacting a transcription reaction mixture comprising a buffer solution comprising Mg²⁺, a DNA template, ribonucleoside tri-phosphates (rNTPs), and RNA polymerase; wherein the rNTPs comprise adenine (A), cytosine (C), guanine (G), and uracil (U) nucleosides, or analogs thereof, at a molar ratio that is within 5-10% of the ratio of A:C:G:T in the DNA template; b) controlling the rate of the transcription reaction of step (a) to be about 10 mM rNTP/hour to about 25 mM rNTP/hour; and c) stopping the transcription reaction by digesting the DNA template with deoxyribonuclease (DNase) or quenching the RNA polymerase with a chelator; thereby producing the single-stranded RNA product.

In some embodiments, the present disclosure is drawn to a method for producing a single-stranded RNA product, the method comprising a) reacting a transcription reaction mixture comprising a buffer solution comprising Mg²⁺, a DNA template, ribonucleoside tri-phosphates (rNTPs), and RNA polymerase; wherein the rNTPs comprise adenine (A), cytosine (C), guanine (G), and uracil (U) nucleosides, or analogs thereof, at a molar ratio that is within 5-10% of the ratio of A:C:G:T in the DNA template, and wherein the transcription reaction results in the depletion of rNTPS, and b) stopping the transcription reaction prior to a point in which the rNTPs have been depleted to between about 0% to 35% of the original amount of rNTPs in the reaction mixture; thereby producing the single-stranded RNA product.

In some embodiments, the present disclosure is drawn to a method for producing a single-stranded RNA product, the method comprising a) reacting a transcription reaction mixture comprising a buffer solution comprising Mg²⁺, a DNA template, ribonucleoside tri-phosphates (rNTPs), and RNA polymerase; wherein the rNTPs comprise adenine (A), cytosine (C), guanine (G), and uracil (U) nucleosides, or analogs thereof, at a molar ratio of about 10:13:13:6 (A:C:G:U); and b) controlling the rate of the transcription reaction of step (a) to be about 10 mM rNTP/hour to about 25 mM rNTP/hour, thereby producing the single-stranded RNA product.

In some embodiments, the molar ratio of the rNTPs is within 5-10% of the ratio of A:C:G:T in the DNA template. In some embodiments, the molar ratio of the rNTPs is within 5.1%, within 5.2%, within 5.3%, within 5.4%, within 5.5%. within 5.6%, within 5.7%, within 5.8%, within 5.9%, within 6.0%, within 6.1%, within 6.2%, within 6.3%, within 6.4%, within 6.5%, within 6.6%, within 6.7%, within 6.8%, within 6.9%, within 7.0%, within 7.1%, within 7.2%, within 7.3%, within 7.4%, within 7.5%, within 7.6%, within 7.7%, within 7.8%, within 7.9%, within 8.0%, within 8.1%, within 8.2%, within 8.3%, within 8.4%, within 8.5%, within 8.6%, within 8.7%, within 8.8%, within 8.9%, within 9.0%, within 9.1%, within 9.2%, within 9.3%, within 9.4%, within 9.5%, within 9.6%, within 9.7%, within 9.8%, within 9.9%, within 10.0% of the ratio of A:C:G:T in the DNA template. The molar ratio may be any value or subrange within the recited ranges, including endpoints.

In some embodiments, the molar ratio of the rNTPs is within 6-10% of the ratio of A:C:G:T in the DNA template. In some embodiments, the molar ratio of the rNTPs is within 7-10% of the ratio of A:C:G:T in the DNA template. In some embodiments, the molar ratio of the rNTPs is within 8-10% of the ratio of A:C:G:T in the DNA template. In some embodiments, the molar ratio of the rNTPs is within 9-10% of the ratio of A:C:G:T in the DNA template. In some embodiments, the molar ratio of the rNTPs is within 5-9% of the ratio of A:C:G:T in the DNA template. In some embodiments, the molar ratio of the rNTPs is within 5-8% of the ratio of A:C:G:T in the DNA template. In some embodiments, the molar ratio of the rNTPs is within 5-7% of the ratio of A:C:G:T in the DNA template. In some embodiments, the molar ratio of the rNTPs is within 5-6% of the ratio of A:C:G:T in the DNA template. In some embodiments, the molar ratio of the rNTPs is within 6-9% of the ratio of A:C:G:T in the DNA template. In some embodiments, the molar ratio of the rNTPs is within 6-8% of the ratio of A:C:G:T in the DNA template. In some embodiments, the molar ratio of the rNTPs is within 6-7% of the ratio of A:C:G:T in the DNA template. In some embodiments, the molar ratio of the rNTPs is within 7-9% of the ratio of A:C:G:T in the DNA template. In some embodiments, the molar ratio of the rNTPs is within 7-8% of the ratio of A:C:G:T in the DNA template. In some embodiments, the molar ratio of the rNTPs is within 8-9% of the ratio of A:C:G:T in the DNA template. The molar ratio may be any value or subrange within the recited ranges, including endpoints.

### In Vitro Transcription

In vitro transcription (IVT) generally consists of a reaction that takes place in a single vessel, typically temperature controlled. IVT reactions require key components but can be enhanced by the use of one or more additives and by controlling various reaction conditions. The process generally involves the use of an engineered DNA template combined with an RNA polymerase and nucleoside triphosphates in a reaction buffer. When required by the applicable RNA polymerase of a reaction, Mg²⁺ ions are also part of the reaction mixture. Once an IVT reaction has been carried out for a sufficient amount of time, the reaction is stopped, and the crude reaction transcript can be separated and purified. The various components, additives, conditions, and other properties of IVT reactions are described in detail below.

In some embodiments, the DNA template is in a solution comprising 0 mM to 1200 mM NaCl to produce a salt-spiked DNA template prior to introduction to the transcription reaction mixture. In some embodiments, the DNA template is in a solution comprising 0 mM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 56 mM, 57 mM, 58 mM, 59 mM, 60 mM, 61 mM, 62 mM, 63 mM, 64 mM, 65 mM, 66 mM, 67 mM, 68 mM, 69 mM, 70 mM, 71 mM, 72 mM, 73 mM, 74 mM, 75 mM, 76 mM, 77 mM, 78 mM, 79 mM, 80 mM, 81 mM, 82 mM, 83 mM, 84 mM, or 85 mM. In some embodiments the DNA template is in a solution comprising anywhere between 85 mM to 1200 mM NaCl to produce a salt-spiked DNA template prior to introduction to the transcription reaction mixture. The solution can comprise molar concentrations that may have any value or subrange within the recited ranges, including endpoints.

In specific embodiments, the NaCl is present in the transcription reaction mixture at a concentration of between about 0 mM and about 85 mM, at a concentration of between about 0 mM and about 70 mM, or at a concentration of between about 0 mM and about 65 mM. In some embodiments, the NaCl is present in the transcription reaction mixture at a concentration of about 0 mM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM, about 50 mM, about 51 mM, about 52 mM, about 53 mM, about 54 mM, about 55 mM, about 56 mM, about 57 mM, about 58 mM, about 59 mM, about 60 mM, about 61 mM, about 62 mM, about 63 mM, about 64 mM, about 65 mM, about 66 mM, about 67 mM, about 68 mM, about 69 mM, about 70 mM, about 71 mM, about 72 mM, about 73 mM, about 74 mM, about 75 mM, about 76 mM, about 77 mM, about 78 mM, about 79 mM, about 80 mM, about 81 mM, about 82 mM, about 83 mM, about 84 mM, or about 85 mM. The NaCl may be present in the transcription reaction mixture at molar concentrations that may have any value or subrange within the recited ranges, including endpoints.

In some embodiments, the concentration of rNTPs in the transcription reaction ranges from about 25 mM to about 55 mM. In some embodiments, the concentration of rNTPs is about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM, about 50 mM, about 51 mM, about 52 mM, about 53 mM, about 54 mM, or about 55 mM. In embodiments, the molar concentration of rNTPs may be any value or subrange within the recited ranges, including endpoints.

In some embodiments, the amount of rNTPs can be increased by 15%.

In some embodiments, the rNTPs comprise adenine (A), cytosine (C), guanine (G), and uracil (U) nucleosides, or analogs thereof, at a molar ratio of about 10:13:13:6 (A:C:G:U).

In embodiments, the concentration of RNA polymerase is about 50 units/μL to about 400 units/μL. In embodiments, the concentration of RNA polymerase is about 50 units/μL to about 60 units/μL, about 60 units/μL to about 70 units/μL, about 70 units/μL to about 80 units/μL, about 80 units/μL to about 90 units/μL, about 90 units/μL to about 100 units/μL, about 100 units/μL to about 110 units/μL, about 110 units/μL to about 120 units/μL, about 120 units/μL to about 130 units/μL, about 130 units/μL to about 140 units/μL, about 140 units/μL to about 150 units/μL, about 150 units/μL to about 160 units/μL, about 160 units/μL to about 170 units/μL, about 170 units/μL to about 180 units/μL, about 180 units/μL to about 190 units/μL, about 190 units/μL to about 200 units/μL, about 200 units/μL to about 210 units/μL, about 210 units/μL to about 220 units/μL, about 220 units/μL to about 230 units/μL, about 230 units/μL to about 240 units/μL, about 240 units/μL to about 250 units/μL, about 250 units/μL to about 260 units/μL, about 260 units/μL to about 270 units/μL, about 270 units/μL to about 280 units/μL, about 280 units/μL to about 290 units/μL, or about 290 units/μL to about 300 units/μL. In specific embodiments, the concentration of RNA polymerase is about 100 units/μL to about 200 units/μL. In specific embodiments, the concentration of RNA polymerase is about 100 units/μL to about 150 units/μL. In embodiments, the range may be any interval recited between the amounts recited herein.

In some embodiments, the in vitro transcription reaction method produces about 10 to about 20 g/L of single-stranded RNA product. In some embodiments, the method produces about 10 g/L, about 11 g/L, about 12 g/L, about 13 g/L, about 14 g/L, about 15 g/L, about 16 g/L, about 17 g/L, about 18 g/L, about 19 g/L, or about 20 g/L of single-stranded RNA product. In embodiments, the method may produce about 14 to about 20 g/L of single-stranded RNA product. In embodiments, the concentration of single-stranded RNA product may be any value or subrange within the recited range, including endpoints.

In some embodiments, the single-stranded RNA product is about 75% to about 100% pure. In some embodiments, the single-stranded RNA product is about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% pure. In some embodiments, the single-stranded RNA product is about 90% to about 100% pure. In some embodiments, the single-stranded RNA product is about 91% to about 100% pure. In some embodiments, the single-stranded RNA product is about 92% to about 100% pure. In some embodiments, the single-stranded RNA product is about 93% to about 100% pure. In some embodiments, the single-stranded RNA product is about 94% to about 100% pure. In some embodiments, the single-stranded RNA product is about 95% to about 100% pure. In some embodiments, the single-stranded RNA product is about 96% to about 100% pure. In some embodiments, the single-stranded RNA product is about 97% to about 100% pure. In some embodiments, the single-stranded RNA product is about 98% to about 100% pure. In some embodiments, the single-stranded RNA product is about 99% to about 100% pure. In some embodiments, the single-stranded RNA product is about 99.1%, about 99.2%, about 99.3%, about 99.4%, about 99.5%, about 99.6%, about 99.7%, about 99.8%, about 99.9%, or about 100% pure. In embodiments, the percentage of purity of the single-stranded RNA product may be any value or subrange within the recited range, including endpoints.

In some embodiments, the rate of reaction is controlled by having the initial total rNTP concentration be in the range of about 10 mM to about 80 mM. In some embodiments, the initial total rNTP concentration may be 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 56 mM, 57 mM, 58 mM, 59 mM, 60 mM, 61 mM, 62 mM, 63 mM, 64 mM, 65 mM, 66 mM, 67 mM, 68 mM, 69 mM, 70 mM, 71 mM, 72 mM, 73 mM, 74 mM, 75 mM, 76 mM, 77 mM, 78 mM, 79 mM, or 80 mM. In embodiments, the initial total rNTP concentration may be any value or subrange within the recited range, including endpoints.

In some embodiments, the rate of the in vitro transcription reaction may be controlled by having the initial total rNTP concentration be in the range of about 20 mM to about 60 mM and the Mg²⁺ concentration be in the range of about 15 mM to about 80 mM.

### Temperature of Reaction

In aspects of the present application, reaction temperature plays a significant role during the in vitro transcription process. Generally, higher temperatures increase the reaction rate and raise the average kinetic energy of the reactants. Further, typical in vitro transcription reactions are performed at room temperature or at 37° C. The rate of transcription decreases considerably when carried out at lower temperatures. Without being bound to any one theory, lower reaction temperatures slow the polymerase's progression, thereby preventing it from being displaced by secondary structure or a string of one specific nucleotide. In embodiments, the reaction temperature during the method of producing a transcribed RNA product is about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., or about 45° C. In embodiments, the reaction temperature during the method of producing a transcribed RNA product is 25°−27° C., 26°−28° C., 27°−29° C., 28°−30° C., 29°−31° C., 30°−32° C., 31°−33° C., 32°−34° C., 33°−35° C., 34°−36° C., 35°−37° C., 36°−38° C., 37°−39° C., 38°−40° C., 39°−41° C., 40°−42° C., 41°−43° C., 42°−44° C., or 43°−45° C. In some embodiments, step (a) of the method, wherein step (a) comprises reacting the transcription reaction mixture, and step (b) of the method, wherein step (b) comprises controlling the rate of the transcription reaction, are each independently conducted at a temperature in a range from about 30° C. to about 40° C.

### Time of Reaction

As used herein “time of reaction” refers to incubation time of the reaction until the reaction is stopped. In aspects of the present application, reaction time plays a significant role in the quality and quantity of RNA produced in an in vitro transcription reaction. For example, the typical reaction time as contemplated herein is 4 hours, however the time may be adjusted to provide maximum results for each desired RNA transcript. For transcripts shorter than 4 kb, reaction or incubation times may only be about 2 to 3 hours. For transcripts longer than 4 kb, reaction times may only be 2 hours to minimize heat exposure that can cause RNA degradation. While typical reaction times may vary in duration, extended overnight incubation is not recommended because at low nucleoside triphosphate concentrations, the T7 RNA polymerase exerts RNase activity.

In embodiments, the reaction time of in vitro transcription contemplated herein is from about 60 minutes to about 240 minutes. In embodiments, the transcription reaction time is at least 60 minutes prior to stopping the reaction. In embodiments, the transcription reaction time is at least 70 minutes prior to stopping the reaction. In embodiments, the transcription reaction time is at least 80 minutes prior to stopping the reaction. In embodiments, the transcription reaction time is at least 90 minutes prior to stopping the reaction. In embodiments, the reaction time is at least 100 minutes prior to stopping the reaction. In embodiments, the reaction time is at least 110 minutes prior to stopping the reaction. In embodiments, the reaction time is at least 120 minutes prior to stopping the reaction. In embodiments, the reaction time is at least 130 minutes prior to stopping the reaction. In embodiments, the reaction time is at least 140 minutes prior to stopping the reaction. In embodiments, the reaction time is at least 150 minutes prior to stopping the reaction. In embodiments, the reaction time is at least 160 minutes prior to stopping the reaction. In embodiments, the reaction time is at least 170 minutes prior to stopping the reaction. In embodiments, the reaction time is at least 180 minutes prior to stopping the reaction. In embodiments, the reaction time is at least 190 minutes prior to stopping the reaction. In embodiments, the reaction time is at least 200 minutes prior to stopping the reaction. In embodiments, the reaction time is at least 210 minutes prior to stopping the reaction. In embodiments, the reaction time is at least 220 minutes prior to stopping the reaction. In embodiments, the reaction time is at least 230 minutes prior to stopping the reaction. In embodiments, the reaction time is at least 240 minutes prior to stopping the reaction. In embodiments, the reaction time is from about 60 minutes to 240 minutes prior to stopping the reaction. In embodiments, the transcription reaction is stopped after about 120 minutes to about 180 minutes. The reaction time may involve any time range or time interval recited between the amounts recited herein.

In some embodiments, the rate of transcription reaction of step (a) is controlled to be about 10 mM rNTP/hour to about 25 mM rNTP/hour. In some embodiments, the rate of transcription reaction of step (a) is controlled to be about 10 mM rNTP/hour, about 11 mM rNTP/hour, about 12 mM rNTP/hour, about 13 mM rNTP/hour, about 14 mM rNTP/hour, about 15 mM rNTP/hour, about 16 mM rNTP/hour, about 17 mM rNTP/hour, about 18 mM rNTP/hour, about 19 mM rNTP/hour, about 20 mM rNTP/hour, about 21 mM rNTP/hour, about 22 mM rNTP/hour, about 23 mM rNTP/hour, about 24 mM rNTP/hour, about 25 mM rNTP/hour. Each of these values may be any value or subrange within the recited ranges, including endpoints.

In some embodiments, the rate of the transcription reaction is controlled to be about 13 mM rNTP/hour to about 20 mM rNTP/hour. In some embodiments, the rate of the transcription reaction is controlled to be about 13 mM rNTP/hour, about 14 mM rNTP/hour, about 15 mM rNTP/hour, about 16 mM rNTP/hour, about 17 mM rNTP/hour, about 18 mM rNTP/hour, about 19 mM rNTP/hour, or about 20 mM rNTP/hour. Each of these values may be any value or subrange within the recited ranges, including endpoints.

In some embodiments, the rate of transcription may be controlled by increasing the concentration of rNTPs. In some embodiments, the rate of transcription may be controlled by decreasing the concentration of RNA polymerase. In some embodiments, the rate of transcription is controlled by increasing the concentration of rNTPs and/or decreasing the concentration of RNA polymerase.

In some embodiments, the rate of transcription may be controlled by changing the temperature, salt concentration, magnesium concentration, pH, or a combination thereof.

In vitro Transcription Termination

As used herein, “in vitro transcription termination” refers to a process of ending or stopping the reaction. Transcription termination occurs when a transcribing RNA polymerase releases the DNA template and the RNA that is being processed. Termination is required for preventing the inappropriate transcription of downstream nucleotides, and for recycling of the polymerase. There are multiple methods to stop transcription known in the art including specific sequences to stop transcription, alternate polymerases, and degrading the template. For example, if a plasmid is linearized, then the T7 polymerase has the propensity to read-through transcription or “run-off.”

In some embodiments, the reaction may be stopped prior to a point in which the rNTPs have been depleted to between about 0% to 35% of the original amount of rNTPs in the reaction mixture. In some embodiments, the reaction may be stopped prior to a point in which the rNTPs have been depleted to about 0%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, or about 35% of the original amount of rNTPs in the reaction mixture. The percentage value may be any value or subrange within the recited ranges, including endpoints.

In some embodiments, the reaction may be stopped prior to a point in which the rNTPs have been depleted to between about 0% to 5% of the original amount of rNTPs in the reaction mixture. In some embodiments, the reaction may be stopped prior to a point in which the rNTPs have been depleted to between about 5% to 10% of the original amount of rNTPs in the reaction mixture. In some embodiments, the reaction may be stopped prior to a point in which the rNTPs have been depleted to between about 10% to 15% of the original amount of rNTPs in the reaction mixture. In some embodiments, the reaction may be stopped prior to a point in which the rNTPs have been depleted to between about 15% to 20% of the original amount of rNTPs in the reaction mixture. In some embodiments, the reaction may be stopped prior to a point in which the rNTPs have been depleted to between about 20% to 25% of the original amount of rNTPs in the reaction mixture. In some embodiments, the reaction may be stopped prior to a point in which the rNTPs have been depleted to between about 25% to 30% of the original amount of rNTPs in the reaction mixture. In some embodiments, the reaction may be stopped prior to a point in which the rNTPs have been depleted to between about 30% to 35% of the original amount of rNTPs in the reaction mixture. The percentage value may be any value or subrange within the recited ranges, including endpoints.

In some embodiments, the purity of the RNA transcript product as determined by measuring the percentage of full-length RNA in the RNA transcript product is increased as compared to an in vitro transcription wherein the transcription reaction was not stopped when the rNTPs have been depleted to about 0% to about 35% of the original amount of rNTPs in the reaction mixture.

In some embodiments, the transcription reaction may be stopped by digesting the DNA template with deoxyribonuclease (DNase) or quenching the RNA polymerase with a chelator; thereby producing the single-stranded RNA product. In some embodiments, the chelator is selected from NTA (nitrilotriacetic acid), EDTA (ethylenediaminetetraacetic acid), EDTPO (ethylenediamine tetra(methylene phosphoric acid)), EGTA (ethylene glycol-bisO-aminoethyl ether)-N,N,N′,N′-tetraacetic acid, BAPTA (1,2-Bis(2-aminophenoxy)ethane-N,N,N′,N′-tetraacetic acid), DFOA (Deferoxamine Mesylate), Dimethoxynitrophenamine (1-(2-Nitro-4,5-dimethoxyphenyl)-1,2-diaminoethane-N,N,N′,N′-tetraacetic Acid)), CDTA (1,2-cyclohexylenedinitrilo)tetraacetic acid), DPTA (diethylenetriaminepentaacetic acid), PIH (pyridoxal isonicotinoyl hydrazone), malonic acid, and TPEN (N′-Tetrakis(2-pyridylmethyl)ethylenediamine).

The present application contemplates digesting or degrading the template as a method of stopping the reaction. In embodiments, the present application contemplates methods to reduce DNA contamination. In embodiments, in vitro transcription can be terminated by adding DNase I. In embodiments, DNase I is used to eliminate all genomic DNA resulting in purified RNA. In embodiments, EDTA is used in combination with DNase I to stop in vitro transcription.

In some embodiments, the transcription reaction is stopped after about 60 minutes to about 240 minutes. In embodiments, the transcription reaction time is at least 60 minutes prior to stopping the reaction. In embodiments, the transcription reaction time is at least 70 minutes prior to stopping the reaction. In embodiments, the transcription reaction time is at least 80 minutes prior to stopping the reaction. In embodiments, the transcription reaction time is at least 90 minutes prior to stopping the reaction. In embodiments, the reaction time is at least 100 minutes prior to stopping the reaction. In embodiments, the reaction time is at least 110 minutes prior to stopping the reaction. In embodiments, the reaction time is at least 120 minutes prior to stopping the reaction. In embodiments, the reaction time is at least 130 minutes prior to stopping the reaction. In embodiments, the reaction time is at least 140 minutes prior to stopping the reaction. In embodiments, the reaction time is at least 150 minutes prior to stopping the reaction. In embodiments, the reaction time is at least 160 minutes prior to stopping the reaction. In embodiments, the reaction time is at least 170 minutes prior to stopping the reaction. In embodiments, the reaction time is at least 180 minutes prior to stopping the reaction. In embodiments, the reaction time is at least 190 minutes prior to stopping the reaction. In embodiments, the reaction time is at least 200 minutes prior to stopping the reaction. In embodiments, the reaction time is at least 210 minutes prior to stopping the reaction. In embodiments, the reaction time is at least 220 minutes prior to stopping the reaction. In embodiments, the reaction time is at least 230 minutes prior to stopping the reaction. In embodiments, the reaction time is at least 240 minutes prior to stopping the reaction. In embodiments, the reaction time is from about 60 minutes to 240 minutes prior to stopping the reaction. In embodiments, the transcription reaction is stopped after about 120 minutes to about 180 minutes.

### Addition of Chelators

In some embodiments, chelators may be added to the transcription reaction mixture to increase the purity and reduce degradation of long transcripts. In some embodiments, the chelator is selected from NTA (nitrilotriacetic acid), EDTA (ethylenediaminetetraacetic acid), EDTPO (ethylenediamine tetra(methylene phosphoric acid)), EGTA (ethylene glycol-bisO-aminoethyl ether)-N,N,N′,N′-tetraacetic acid, BAPTA (1,2-Bis(2-aminophenoxy)ethane-N,N,N′,N′-tetraacetic acid), DFOA (Deferoxamine Mesylate), Dimethoxynitrophenamine (1-(2-Nitro-4,5-dimethoxyphenyl)-1,2-diaminoethane-N,N,N′,N′-tetraacetic Acid)), CDTA (1,2-cyclohexylenedinitrilo)tetraacetic acid), DPTA (diethylenetriaminepentaacetic acid), PIH (pyridoxal isonicotinoyl hydrazone), malonic acid, and TPEN (N′-Tetrakis(2-pyridylmethyl)ethylenediamine).

In some embodiments, chelators may be added to the transcription reaction at a concentration ranging from about 1 mM to about 120 mM. In some embodiments, chelators may be added to the transcription reaction at a concentration ranging from about 5 mM to about 50 mM.

In specific embodiments, EDTPO or EDTA may be added to the transcription reaction at a concentration ranging from about 5 mM to about 50 mM. In some embodiments, EDTPO or EDTA may be added to the transcription reaction at a concentration of about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM, or about 50 mM.

In specific embodiments, NTA or EGTA may be added to the transcription reaction at a concentration ranging from about 1 mM to about 120 mM. In some embodiments, NTA or EGTA may be added to the transcription reaction at a concentration of about 1 mM to about 10 mM, about 11 mM to about 20 mM, about 21 mM to about 30 mM, about 31 mM to about 40 mM, about 41 mM to about 50 mM, about 51 mM to about 60 mM, about 61 mM to about 70 mM, about 71 mM to about 80 mM, about 81 mM to about 90 mM, about 91 mM to about 100 mM, about 101 mM to about 110 mM, about 111 mM to about 120 mM, about 121 mM to about 130 mM, about 131 mM to about 140 mM, about 141 mM to about 150 mM, about 151 mM to about 160 mM, about 161 mM to about 170 mM, about 171 mM to about 180 mM, about 181 mM to about 190 mM, or about 191 mM to about 200 mM. Each of the values or ranges recited herein may include any value or subrange therebetween, including endpoints.

In specific embodiments, NTA or EGTA may be added to the transcription reaction at a concentration of about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM, about 50 mM, about 51 mM, about 52 mM, about 53 mM, about 54 mM, about 55 mM, about 56 mM, about 57 mM, about 58 mM, about 59 mM, about 60 mM, about 61 mM, about 62 mM, about 63 mM, about 64 mM, about 65 mM, about 66 mM, about 67 mM, about 68 mM, about 69 mM, about 70 mM, about 71 mM, about 72 mM, about 73 mM, about 74 mM, about 75 mM, about 76 mM, about 77 mM, about 78 mM, about 79 mM, about 80 mM, about 81 mM, about 82 mM, about 83 mM, about 84 mM, about 85 mM, about 86 mM, about 87 mM, about 88 mM, about 89 mM, about 90 mM, about 91 mM, about 92 mM, about 93 mM, about 94 mM, about 95 mM, about 96 mM, about 97 mM, about 98 mM, about 99 mM, about 100 mM, about 101 mM, about 102 mM, about 103 mM, about 104 mM, about 105 mM, about 106 mM, about 107 mM, about 108 mM, about 109 mM, about 110 mM, about 111 mM, about 112 mM, about 113 mM, about 114 mM, about 115 mM, about 116 mM, about 117 mM, about 118 mM, about 119 mM, about 120 mM, about 121 mM, about 122 mM, about 123 mM, about 124 mM, about 125 mM, about 126 mM, about 127 mM, about 128 mM, about 129 mM, about 130 mM, about 131 mM, about 132 mM, about 133 mM, about 134 mM, about 135 mM, about 136 mM, about 137 mM, about 138 mM, about 139 mM, about 140 mM, about 141 mM, about 142 mM, about 143 mM, about 144 mM, about 145 mM, about 146 mM, about 147 mM, about 148 mM, about 149 mM, about 150 mM, about 151 mM, about 152 mM, about 153 mM, about 154 mM, about 155 mM, about 156 mM, about 157 mM, about 158 mM, about 159 mM, about 160 mM, about 161 mM, about 162 mM, about 163 mM, about 164 mM, about 165 mM, about 166 mM, about 167 mM, about 168 mM, about 169 mM, about 170 mM, about 171 mM, about 172 mM, about 173 mM, about 174 mM, about 175 mM, about 176 mM, about 177 mM, about 178 mM, about 179 mM, about 180 mM, about 181 mM, about 182 mM, about 183 mM, about 184 mM, about 185 mM, about 156 mM, about 187 mM, about 188 mM, about 189 mM, about 190 mM, about 191 mM, about 192 mM, about 193 mM, about 194 mM, about 195 mM, about 196 mM, about 197 mM, about 198 mM, about 199 mM, or about 200 mM.

Aside from alternative chelators, in some embodiments one may spike in more chelator as the IVT reaction proceeds. Without being bound by theory, more chelator can theoretically be added without significantly inhibiting the IVT reaction, and therefore potentially improve RNA integrity in the IVT. In other embodiments, one may replace the buffer of the IVT with a buffer that can also chelate magnesium.

In yet additional embodiments, one may attach a chelator, e.g., NTA, to a solid substrate like beads packed in a column. This column charged with magnesium would theoretically offer a continuous flow IVT system where magnesium is sequestered on the column where the IVT reaction proceeds. The RNA will then exit the column free of magnesium, potentially improving integrity of RNA. NTA columns such as the one described are commonly used for affinity chromatography of histidine-tagged proteins.

In some embodiments, long transcripts may be about 4,000 nucleotides in length or longer. In specific embodiments, transcripts may be 4,000 to 15,000 nucleotides in length. In specific embodiments, transcripts may be 4,000 to 4,500 nucleotides in length. In some embodiments, transcripts may be 4,500 to 5,000 nucleotides in length. In some embodiments, transcripts may be 5,000 to 5,500 nucleotides in length. In some embodiments, transcripts may be 5,500 to 6,000 nucleotides in length. In some embodiments, transcripts may be 6,000 to 6,500 nucleotides in length. In some embodiments, transcripts may be 6,500 to 7,000 nucleotides in length. In some embodiments, transcripts may be 7,000 to 7,500 nucleotides in length. In some embodiments, transcripts may be 7,500 to 8,000 nucleotides in length. In some embodiments, transcripts may be 8,000 to 8,500 nucleotides in length. In some embodiments, transcripts may be 8,500 to 9,000 nucleotides in length. In some embodiments, transcripts may be 9,000 to 9,500 nucleotides in length. In some embodiments, transcripts may be 9,500 to 10,000 nucleotides in length. In some embodiments, transcripts may be 10,000 to 10,500 nucleotides in length. In some embodiments, transcripts may be 10,500 to 11,000 nucleotides in length. In some embodiments, transcripts may be 11,000 to 11,500 nucleotides in length. In some embodiments, transcripts may be 11,500 to 12,000 nucleotides in length. In some embodiments, transcripts may be 12,000 to 12,500 nucleotides in length. In some embodiments, transcripts may be 12,500 to 13,000 nucleotides in length. In some embodiments, transcripts may be 13,000 to 13,500 nucleotides in length. In some embodiments, transcripts may be 13,500 to 14,000 nucleotides in length. In some embodiments, transcripts may be 14,000 to 14,500 nucleotides in length. In some embodiments, transcripts may be 14,500 to 15,000 nucleotides in length.

In some embodiments, transcripts may be 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000, 10,100, 10,200, 10,300, 10,400, 10,500, 10,600, 10,700, 10,800, 10,900, 10,000, 11,100, 11,200, 11,300, 11,400, 11,500, 11,600, 11,700, 11,800, 11,900, 12,000, 12,100, 12,200, 12,300, 12,400, 12,500, 12,600, 12,700, 12,800, 12,900, 13,000, 13,100, 13,200, 13,300, 13,400, 13,500, 13,600, 13,700, 13,800, 13,900, 14,000, 14,100, 14,200, 14,300, 14,400, 14,500, 14,600, 14,700, 14,800, 14,900, 15,000, nucleotides in length, or longer.

### Compositions

### Buffers

Any suitable buffer composition can be used in an IVT process of the present disclosure. Generally, the buffer system selected for an IVT process is one that can mimic a biological environment for the enzymes used in the process and that can further facilitate the transcription reaction. Suitable buffers include, without limitation, phosphate-buffered saline (PBS) 2-(N-Morpholino)ethanesulfonic acid (MES), 2-Amino-2-hydroxymethyl-propane-1,3-diol hydrochloric acid (Tris or Tris-HCl), 4-(2-hydroxyethyl)-1-piperazineethanesulfonic acid (HEPES), 2-Bis(2-hydroxyethyl)amino-2-(hydroxymethyl)-1,3-propanediol (Bis-Tris), N-(2-Hydroxy-1,1-bis(hydroxymethyl)ethyl)glycine (Tricine), 3-morpholinopropane-1-sulfonic acid (MOPS), acetate, citrate, saline sodium citrate (SSC), phosphate, N-Cyclohexyl-2-aminoethanesulfonic acid (CHES), borate, [tris(hydroxymethyl)methylamino]propanesulfonic acid (TAPS), 2-(bis(2-hydroxyethyl)amino)acetic acid (Bicine), 3-[N-tris(hydroxymethyl)methylamino]-2-hydroxypropanesulfonic acid (TAPSO), 2-[[1,3-dihydroxy-2-(hydroxymethyl)propan-2-yl]amino]ethanesulfonic acid (TES), malonate, or piperazine-N,N′-bis(2-ethanesulfonic acid (PIPES).

The buffer can also be in any suitable concentration. In embodiments, the buffer is in a concentration in the range of about 10 mM to about 2000 mM, about 75 mM to about 1800 mM, about 80 mM to about 1700 mM, about 85 mM to about 1600 mM, about 90 mM to about 1500 mM, about 95 mM to about 1400 mM, about 100 mM to about 1300 mM, about 125 mM to about 1200 mM, about 150 mM to about 1100 mM, about 175 mM to about 1000 mM, about 200 mM to about 900 mM, about 250 mM to about 800 mM, about 275 mM to about 700 mM, about 300 mM to about 600 mM, about 325 mM to about 550 mM, about 350 mM to about 525 mM, about 325 mM to about 575 mM, about 350 mM to about 450 mM, or about 200 mM to about 600 mM. In some embodiments, the buffer is in a concentration in the range of about 10 mM to about 200 mM. In some embodiments, the buffer is in a concentration of about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM, about 50 mM, about 51 mM, about 52 mM, about 53 mM, about 54 mM, about 55 mM, about 56 mM, about 57 mM, about 58 mM, about 59 mM, about 60 mM, about 61 mM, about 62 mM, about 63 mM, about 64 mM, about 65 mM, about 66 mM, about 67 mM, about 68 mM, about 69 mM, about 70 mM, about 71 mM, about 72 mM, about 73 mM, about 74 mM, about 75 mM, about 76 mM, about 77 mM, about 78 mM, about 79 mM, about 80 mM, about 81 mM, about 82 mM, about 83 mM, about 84 mM, about 85 mM, about 86 mM, about 87 mM, about 88 mM, about 89 mM, about 90 mM, about 91 mM, about 92 mM, about 93 mM, about 94 mM, about 95 mM, about 96 mM, about 97 mM, about 98 mM, about 99 mM, about 100 mM, about 101 mM, about 102 mM, about 103 mM, about 104 mM, about 105 mM, about 106 mM, about 107 mM, about 108 mM, about 109 mM, about 110 mM, about 111 mM, about 112 mM, about 113 mM, about 114 mM, about 115 mM, about 116 mM, about 117 mM, about 118 mM, about 119 mM, about 120 mM, about 121 mM, about 122 mM, about 123 mM, about 124 mM, about 125 mM, about 126 mM, about 127 mM, about 128 mM, about 129 mM, about 130 mM, about 131 mM, about 132 mM, about 133 mM, about 134 mM, about 135 mM, about 136 mM, about 137 mM, about 138 mM, about 139 mM, about 140 mM, about 141 mM, about 142 mM, about 143 mM, about 144 mM, about 145 mM, about 146 mM, about 147 mM, about 148 mM, about 149 mM, about 150 mM, about 151 mM, about 152 mM, about 153 mM, about 154 mM, about 155 mM, about 156 mM, about 157 mM, about 158 mM, about 159 mM, about 160 mM, about 161 mM, about 162 mM, about 163 mM, about 164 mM, about 165 mM, about 166 mM, about 167 mM, about 168 mM, about 169 mM, about 170 mM, about 171 mM, about 172 mM, about 173 mM, about 174 mM, about 175 mM, about 176 mM, about 177 mM, about 178 mM, about 179 mM, about 180 mM, about 181 mM, about 182 mM, about 183 mM, about 184 mM, about 185 mM, about 186 mM, about 187 mM, about 188 mM, about 189 mM, about 190 mM, about 191 mM, about 192 mM, about 193 mM, about 194 mM, about 195 mM, about 196 mM, about 197 mM, about 198 mM, about 199 mM, about 200 mM. Each of the values or ranges recited herein may include any value or subrange therebetween, including endpoints.

In embodiments, the buffer can be in a concentration of about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 375 mM, about 376 mM, about 377 mM, about 378 mM, about 379 mM, about 380 mM, about 381 mM, about 382 mM, about 383 mM, about 384 mM, about 385 mM, about 386 mM, about 387 mM, about 388 mM, about 389 mM, about 390 mM, about 391 mM, about 392 mM, about 392 mM, about 393 mM, about 394 mM, about 395 mM, about 396 mM, about 397 mM, about 398 mM, about 399 mM, about 400 mM, about 401 mM, about 402 mM, about 403 mM, about 404 mM, about 405 mM, about 406 mM, about 407 mM, about 408 mM, about 409 mM, about 410 mM, about 411 mM, about 412 mM, about 413 mM, about 414 mM, about 415 mM, about 416 mM, about 417 mM, about 418 mM, about 419 mM, about 420 mM, about 421 mM, about 422 mM about 422 mM, about 423 mM, about 424 mM, about 425 mM, about 430 mM, about 440 mM, about 450 mM, about 460 mM, about 470 mM, about 480 mM, about 490 mM, about 500 mM, about 510 mM, about 520 mM, about 530 mM, about 540 mM, about 550 mM, about 560 mM, about 570 mM, about 580 mM, about 590 mM, or about 600 mM.

In some embodiments, the initial pH of the reaction mixture is in a range from about 6.5 to about 8.0. In some embodiments, the initial pH of the reaction mixture is about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, or about 8.0. In some embodiments, the pH value may be any value or subrange within the recited range, including endpoints.

### Nucleoside Triphosphates (NTPs)

RNA synthesis is catalyzed by RNA polymerase, which covalently links the free —OH group on the 3′ carbon of a growing chain of nucleotides to the α-phosphate on the 5′ carbon of the next NTP, releasing the β- and γ-phosphate groups as pyrophosphate (PPi). This results in a phosphodiester linkage between the two NTPs. The release of PPi provides the energy necessary for the reaction to occur. Typically, the NTPs in a transcription reaction are the four natural ribonucleoside triphosphates, adenosine triphosphate (ATP), uridine triphosphate (UTP), guanosine triphosphate (GTP), and cytosine triphosphate (CTP). Generally, when an in vitro transcription reaction is described in the present disclosure as being performed with NTPs without any further description, it is understood that such reaction is being carried out in the presence of ATP, UTP, GTP, and CTP. However, IVT reactions can also be carried out in the presence of one or more modified nucleoside triphosphates. For a given base-type (i.e., A, U, G, C) of an RNA transcript prepared by IVT, the transcript can be prepared in any desired molar ratio of NTPs or modified NTPs for that base-type. For example, the U-bases of an in vitro transcribed RNA can comprise 50% of natural uridine and 50% 5-methoxy uridine.

In embodiments, the IVT reactions of the present disclosure include non-natural, modified, and chemically modified nucleotides, including any such nucleotides known in the art. Nucleotides can be artificially modified at either the base portion or the sugar portion. In nature, most polynucleotides comprise nucleotides that are “unmodified” or “natural” nucleotides, which include the purine bases adenine (A) and guanine (G), and the pyrimidine bases thymine (T), cytosine (C) and uracil (U). These bases are typically fixed to a ribose at the 1′ position. The use of RNA polynucleotides comprising chemically modified nucleotides have been shown to improve RNA expression, expression rates, half-life and/or expressed protein concentrations. RNA polynucleotides comprising chemically modified nucleotides have also been useful in optimizing protein localization thereby avoiding deleterious bio-responses such as immune responses and/or degradation pathways.

In embodiments, one or more of the nucleoside triphosphates can be chemically modified.

Examples of modified or chemically modified nucleotides include 5-hydroxycytidines, 5-alkylcytidines, 5-hydroxyalkylcytidines, 5-carboxycytidines, 5-formylcytidines, 5-alkoxycytidines, 5-alkynylcytidines, 5-halocytidines, 2-thiocytidines, N4-alkylcytidines, N4-aminocytidines, N4-acetylcytidines, and N4,N4-dialkylcytidines.

Examples of modified or chemically-modified nucleotides include 5-hydroxycytidine, 5-methylcytidine, 5-hydroxymethylcytidine, 5-carboxycytidine, 5-formylcytidine, 5-methoxycytidine, 5-propynylcytidine, 5-bromocytidine, 5-iodocytidine, 2-thiocytidine; N4-methylcytidine, N4-aminocytidine, N4-acetylcytidine, and N4,N4-dimethylcytidine.

Examples of modified or chemically modified nucleotides include 5-hydroxyuridines, 5-alkyluridines, 5-hydroxyalkyluridines, 5-carboxyuridines, 5-carboxyalkylesteruridines, 5-formyluridines, 5-alkoxyuridines, 5-alkynyluridines, 5-halouridines, 2-thiouridines, and 6-alkyluridines.

Examples of modified or chemically modified nucleotides include 5-hydroxyuridine, 5-methyluridine, 5-hydroxymethyluridine, 5-carboxyuridine, 5-carboxymethylesteruridine, 5-formyluridine, 5-methoxyuridine (also referred to herein as “5MeOU”), 5-propynyluridine, 5-bromouridine, 5-fluorouridine, 5-iodouridine, 2-thiouridine, and 6-methyluridine.

Examples of modified or chemically modified nucleotides include 5-methoxycarbonylmethyl-2-thiouridine, 5-methylaminomethyl-2-thiouridine, 5-carbamoylmethyluridine, 5-carbamoylmethyl-2′-O-methyluridine, 1-methyl-3-(3-amino-3-carboxypropy)pseudouridine, 5-methylaminomethyl-2-selenouridine, 5-carboxymethyluridine, 5-methyldihydrouridine, 5-taurinomethyluridine, 5-taurinomethyl-2-thiouridine, 5-(isopentenylaminomethyl)uridine, 2′-O-methylpseudouridine, 2-thio-2′O-methyluridine, and 3,2′-O-dimethyluridine.

Examples of modified or chemically modified nucleotides include N6-methyladenosine, 2-aminoadenosine, 3-methyladenosine, 8-azaadenosine, 7-deazaadenosine, 8-oxoadenosine, 8-bromoadenosine, 2-methylthio-N6-methyladenosine, N6-isopentenyladenosine, 2-methylthio-N6-isopentenyladenosine, N6-(cis-hydroxyisopentenyl)adenosine, 2-methylthio-N6-(cis-hydroxyisopentenyl)adenosine, N6-glycinylcarbamoyladenosine, N6-threonylcarbamoyl-adenosine, N6-methyl-N6-threonylcarbamoyl-adenosine, 2-methylthio-N6-threonylcarbamoyl-adenosine, N6,N6-dimethyladenosine, N6-hydroxynorvalylcarbamoyladenosine, 2-methylthio-N6-hydroxynorvalylcarbamoyl-adenosine, N6-acetyl-adenosine, 7-methyl-adenine, 2-methylthio-adenine, 2-methoxy-adenine, alpha-thio-adenosine, 2′-O-methyl-adenosine, N6,2′-O-dimethyl-adenosine, N6,N6,2′-O-trimethyl-adenosine, 1,2′-O-dimethyl-adenosine, 2′-O-ribosyladenosine, 2-amino-N6-methyl-purine, 1-thio-adenosine, 2′-F-ara-adenosine, 2′-F-adenosine, 2′-OH-ara-adenosine, and N6-(19-amino-pentaoxanonadecyl)-adenosine.

Examples of modified or chemically modified nucleotides include N1-alkylguanosines, N2-alkylguanosines, thienoguanosines, 7-deazaguanosines, 8-oxoguanosines, 8-bromoguanosines, O6-alkylguanosines, xanthosines, inosines, and N1-alkylinosines.

Examples of modified or chemically modified nucleotides include N1-methylguanosine, N2-methylguanosine, thienoguanosine, 7-deazaguanosine, 8-oxoguanosine, 8-bromoguanosine, O6-methylguanosine, xanthosine, inosine, and N1-methylinosine.

Examples of modified or chemically modified nucleotides include pseudouridines. Examples of pseudouridines include N1-alkylpseudouridines, N1-cycloalkylpseudouridines, N1-hydroxypseudouridines, N1-hydroxyalkylpseudouridines, N1-phenylpseudouridines, N1-phenylalkylpseudouridines, N1-aminoalkylpseudouridines, N3-alkylpseudouridines, N6-alkylpseudouridines, N6-alkoxypseudouridines, N6-hydroxypseudouridines, N6-hydroxyalkylpseudouridines, N6-morpholinopseudouridines, N6-phenylpseudouridines, and N6-halopseudouridines. Examples of pseudouridines include N1-alkyl-N6-alkylpseudouridines, N1-alkyl-N6-alkoxypseudouridines, N1-alkyl-N6-hydroxypseudouridines, N1-alkyl-N6-hydroxyalkylpseudouridines, N1-alkyl-N6-morpholinopseudouridines, N1-alkyl-N6-phenylpseudouridines, and N1-alkyl-N6-halopseudouridines. In these examples, the alkyl, cycloalkyl, and phenyl substituents may be unsubstituted, or further substituted with alkyl, halo, haloalkyl, amino, or nitro substituents.

Examples of pseudouridines include N1-methylpseudouridine (also referred to herein as “N1MPU”), N1-ethylpseudouridine, N1-propylpseudouridine, N1-cyclopropylpseudouridine, N1-phenylpseudouridine, N1-aminomethylpseudouridine, N3-methylpseudouridine, N1-hydroxypseudouridine, and N1-hydroxymethylpseudouridine.

Examples of nucleic acid monomers include modified and chemically modified nucleotides, including any such nucleotides known in the art.

Examples of modified and chemically modified nucleotide monomers include any such nucleotides known in the art, for example, 2′-O-methyl ribonucleotides, 2′-O-methyl purine nucleotides, 2′-deoxy-2′-fluoro ribonucleotides, 2′-deoxy-2′-fluoro pyrimidine nucleotides, 2′-deoxy ribonucleotides, 2′-deoxy purine nucleotides, universal base nucleotides, 5-C-methyl-nucleotides, and inverted deoxyabasic monomer residues.

Examples of modified and chemically modified nucleotide monomers include 3′-end stabilized nucleotides, 3′-glyceryl nucleotides, 3′-inverted abasic nucleotides, and 3′-inverted thymidine.

Examples of modified and chemically modified nucleotide monomers include locked nucleic acid nucleotides (LNA), 2′-O,4′-C-methylene-(D-ribofuranosyl) nucleotides, 2′-methoxyethoxy (MOE) nucleotides, 2′-methyl-thio-ethyl, 2′-deoxy-2′-fluoro nucleotides, and 2′-O-methyl nucleotides. In an exemplary embodiment, the modified monomer is a locked nucleic acid nucleotide (LNA).

Examples of modified and chemically modified nucleotide monomers include 2′,4′-constrained 2′-O-methoxyethyl (cMOE) and 2′-O-Ethyl (cEt) modified DNAs.

Examples of modified and chemically modified nucleotide monomers include 2′-amino nucleotides, 2′-O-amino nucleotides, 2′-C-allyl nucleotides, and 2′-O-allyl nucleotides.

Examples of modified and chemically modified nucleotide monomers include N6-methyladenosine nucleotides.

Examples of modified and chemically modified nucleotide monomers include nucleotide monomers with modified bases 5-(3-amino)propyluridine, 5-(2-mercapto)ethyluridine, 5-bromouridine; 8-bromoguanosine, or 7-deazaadenosine.

Examples of modified and chemically modified nucleotide monomers include 2′-O-aminopropyl substituted nucleotides.

Examples of modified and chemically modified nucleotide monomers include replacing the 2′—OH group of a nucleotide with a 2′-R, a 2′-OR, a 2′-halogen, a 2′-SR, or a 2′-amino, where R can be H, alkyl, alkenyl, or alkynyl.

Exemplary base modifications described above can be combined with additional modifications of nucleoside or nucleotide structure, including sugar modifications and linkage modifications. Certain modified or chemically modified nucleotide monomers may be found in nature.

Preferred nucleotide modifications include N1-methylpseudouridine and 5-methoxyuridine.

In some embodiments, the concentration of rNTPs in the transcription reaction ranges from about 25 mM to about 55 mM. In some embodiments, the concentration of rNTPs is about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM, about 50 mM, about 51 mM, about 52 mM, about 53 mM, about 54 mM, or about 55 mM. In embodiments, the molar concentration of rNTPs may be any value or subrange within the recited ranges, including endpoints.

In some embodiments, the amount of NTPs can be increased by 15%.

In some embodiments, the ratio of adenine nucleosides, guanine nucleosides, cytosine nucleosides, and uracil nucleosides are tuned to be within 0-10% of the ratio of adenine, guanine, cytosine, and thymine bases on the linear DNA template. For example, if 25% of the nucleosides in the linear DNA template were adenine, then the concentration of the rATP in the reaction mixture would be tuned to be from 15% to 35% of all rNTPs in the reaction mixture. In some embodiments, the ratio of adenine nucleosides, guanine nucleosides, cytosine nucleosides, and uracil nucleosides are tuned to be within 0%, 1%, 2%, 3%, 4%, 5%, 6%, 7%, 8%, 9%, or 10% of the ratio of adenine, guanine, cytosine, and thymine bases on the linear DNA template.

In some embodiments, a yield of RNA transcript is increased by at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 45%, at least 46%, at least 47%, at least 48%, at least 49%, at least 50%, as compared to a transcription reaction in which the ratio of adenine nucleosides, guanine nucleosides, cytosine nucleosides, and uracil nucleosides has not been tuned to be within 5% of the ratio of adenine, guanine, cytosine, and thymine bases on the linear DNA template.

In some embodiments, the rNTPs comprise adenine (A), cytosine (C), guanine (G), and uracil (U) nucleosides, or analogs thereof, at a molar ratio that is within about 5% of about 10:13:13:6 (A:C:G:U).

In some embodiments, the rate of transcription may be controlled by increasing the concentration of rNTPs. In some embodiments, the rate of transcription is controlled by increasing the concentration of rNTPs and/or decreasing the concentration of RNA polymerase.

5′ mRNA Caps

Only those RNA molecules that carry the Cap structure are active in Cap dependent translation; “decapitation” of mRNA results in an almost complete loss of their template activity for protein synthesis (Nature, 255:33-37, (1975); J. Biol. Chem., vol. 253:5228-5231, (1978); and Proc. Natl. Acad. Sci. USA, 72:1189-1193, (1975)).

Another element of eukaryotic mRNA is the presence of 2′-O-methyl nucleoside residues at transcript position 1 (Cap 1), and in some cases, at transcript positions 1 and 2 (Cap 2). The 2′-O-methylation of mRNA provides higher efficacy of mRNA translation in vivo (Proc. Natl. Acad. Sci. USA, 77:3952-3956 (1980)) and further improves nuclease stability of the 5′-capped mRNA. The mRNA with Cap 1 (and Cap 2) is a distinctive mark that allows cells to recognize the bona fide mRNA 5′ end, and in some instances, to discriminate against transcripts emanating from infectious genetic elements (Nucleic Acid Research 43: 482-492 (2015)).

Some examples of 5′ cap structures and methods for preparing mRNAs comprising the same are given in WO2015/051169A2, WO/2015/061491, US 2018/0273576, and U.S. Pat. Nos. 8,093,367, 8,304,529, and 10,487,105. Such methods can include cotranscriptional capping in which an RNA capping reagent that hybridizes with the linear DNA template at or near the transcription initiation site is included in the in vitro transcription reaction mixture. Another method is to have the capping done post-transcriptionally through the use of enzymes that can add a cap or methylate certain nucleotides.

In embodiments, RNA transcripts produced by the methods provided herein further comprise a 5′ cap. Any 5′ cap can be included in such RNA molecules. In further embodiments, an RNA cap may be selected from m7GpppA, m7GpppC; unmethylated cap analogs (e.g., GpppG); dimethylated cap analog (e.g., m2,7GpppG), a trimethylated cap analog (e.g., m2,2,7GpppG), dimethylated symmetrical cap analogs (e.g., m7Gpppm7G), or anti reverse cap analogs (e.g., ARCA; m7, 2′OmeGpppG, m72′dGpppG, m7,3′OmeGpppG, m7,3′dGpppG and their tetraphosphate derivatives) (see, e.g., Jemielity, J. et al., RNA 9: 1108-1122 (2003)). In additional embodiments, an RNA cap may be an ARCA cap (3′-OMe-m7G(5′)pppG). The RNA cap may be an mCAP (m7G(5′)ppp(5′)G, N7-Methyl-Guanosine-5′-Triphosphate-5′-Guanosine). The RNA cap may be resistant to hydrolysis.

### Magnesium Ion (Mg²⁺)

Generally, enzymes that have nucleotidyl transferase activity employ a general two-metal-ion mechanism to carry out an NTP condensation reaction as the NTP is adding to the elongating nucleotide strand. (Svetlov, Vladimir, and Evgeny Nudler. “Basic mechanism of transcription by RNA polymerase II.” Biochimica et Biophysica Acta vol. 1829,1 (2013): 20-8). For RNA polymerases that are used in DNA-dependent RNA transcription, the central feature of this mechanism is the employment of two magnesium cations, coordinated by at least two aspartate residues located in the active site. According to this general model the first magnesium (A) promotes deprotonation of the RNA 3′OH, facilitating 3′O-attack on the substrate NTP α-phosphate, which in turn leads to formation of a new phosphodiester bond and a leaving group, pyrophosphate (PPi). Thus, magnesium cations play an essential role in the transcription reaction.

For IVT, any suitable, water-soluble Mg²⁺ salt can be used. For example, the Mg²⁺ can be in the form of aqueous MgCl₂H₃O₂ (i.e., magnesium acetate or MgOAc), MgCl₂, MgI₂, MgBr₂, and Mg(NO₃)₂. In embodiments of the IVT methods of the present disclosure, the concentration of Mg²⁺ in the transcription reaction is determined as an amount relative to the total concentration of NTPs (plus initiating cap if present). The concentration of Mg²⁺ can be in a range of about 1 mM to about 100 mM. In some embodiments, the concentration of Mg²⁺ can be 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 56 mM, 57 mM, 58 mM, 59 mM, 60 mM, 61 mM, 62 mM, 63 mM, 64 mM, 65 mM, 66 mM, 67 mM, 68 mM, 69 mM, 70 mM, 71 mM, 72 mM, 73 mM, 74 mM, 75 mM, 76 mM, 77 mM, 78 mM, 79 mM, 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, or 100 mM. In embodiments, the molar concentration of Mga'⁰ may be any value or subrange within the recited ranges, including endpoints.

### DNA-Dependent RNA Polymerases

Any suitable DNA-dependent RNA polymerase can be used in the IVT methods of the present disclosure. Those skilled in the art will understand that each RNA polymerase will require a specifically matched promoter sequence on the complementary L.DNA strand to direct the RNA polymerase where to begin transcription. In order to synthesize RNA, especially large amounts of RNA, bacteriophage DNA-dependent RNA polymerase (an enzyme) is used to catalyze the transcription of RNA from a DNA template. In embodiments a Phage RNA polymerase is used. In embodiments, the RNA polymerase may be, but is not limited to T7, T3, or SP6. Bacteriophage T7 RNA polymerase is the “prototype” for other DNA-dependent RNA polymerases such as T3, SP6, and mitochondrial DNA-dependent RNA polymerases. It is also considered one of the simplest enzymes catalyzing RNA synthesis. In embodiments, the RNA polymerase is a T7 polymerase. In embodiments, the RNA polymerase is a T7 polymerase. In embodiments, the RNA polymerase is a SP6 polymerase. In embodiments, the RNA polymerase is an E. coli polymerase.

In embodiments, the concentration of RNA polymerase in the IVT reaction is about 50 units/μL to about 400 units/μL. In embodiments, the concentration of RNA polymerase is about 50 units/μL to about 60 units/μL, about 60 units/μL to about 70 units/μL, about 70 units/μL to about 80 units/μL, about 80 units/μL to about 90 units/μL, about 90 units/μL to about 100 units/μL, about 100 units/μL to about 110 units/μL, about 110 units/μL to about 120 units/μL, about 120 units/μL to about 130 units/μL, about 130 units/μL to about 140 units/μL, about 140 units/μL to about 150 units/μL, about 150 units/μL to about 160 units/μL, about 160 units/μL to about 170 units/μL, about 170 units/μL to about 180 units/μL, about 180 units/μL to about 190 units/μL, about 190 units/μL to about 200 units/μL, about 200 units/μL to about 210 units/μL, about 210 units/μL to about 220 units/μL, about 220 units/μL to about 230 units/μL, about 230 units/μL to about 240 units/μL, about 240 units/μL to about 250 units/μL, about 250 units/μL to about 260 units/μL, about 260 units/μL to about 270 units/μL, about 270 units/μL to about 280 units/μL, about 280 units/μL to about 290 units/μL, or about 290 units/μL to about 300 units/μL. In specific embodiments, the concentration of RNA polymerase is about 100 units/μL to about 200 units/μL. In specific embodiments, the concentration of RNA polymerase is about 100 units/μL to about 150 units/μL. In embodiments, the range may be any interval recited between the amounts recited herein.

In some embodiments, the RNA polymerase KU activity per mL of transcription reaction mixture is greater than or equal to 125 KU RNA polymerase activity.

In some embodiments, the rate of transcription may be controlled by decreasing the concentration of RNA polymerase. In some embodiments, the rate of transcription is controlled by increasing the concentration of rNTPs and/or decreasing the concentration of RNA polymerase.

### DNA Linearization

As used herein, “L.DNA” refers to linearized DNA. Generally, DNA linearization is a method to produce RNA transcripts of a specified length. The DNA plasmid used as the template is linearized by a restriction enzyme downstream from the insert. In embodiments, restriction enzymes that generate 5′-overhangs may be used, and preferred, versus 3′-overhangs. Because RNA polymerases tend to “read-through” transcription, circular plasmid templates generate long heterogeneous RNA transcripts in higher quantities than linear templates. Accordingly, DNA plasmids must be completely linearized to ensure efficient synthesis of specified RNA transcript lengths. In embodiments, the application refers to “L.DNA template,” which refers to the plasmid DNA used as a template which has been linearized.

In embodiments, the amount of L.DNA template in the transcription reaction mixture is from about 0.01 mg/mL to about 0.5 mg/mL. In embodiments, the amount of L.DNA template in the transcription reaction mixture is from about 0.01 mg/mL to about 0.3 mg/mL In embodiments, the amount of L.DNA template in the transcription reaction mixture is about 0.01 mg/mL, about 0.02 mg/mL, about 0.03 mg/mL, about 0.04 mg/mL, about 0.05 mg/mL, about 0.06 mg/mL, about 0.07 mg/mL, about 0.08 mg/mL, about 0.09 mg/mL, about 0.10 mg/mL, about 0.11 mg/mL, about 0.12 mg/mL, about 0.13 mg/mL, about 0.14 mg/mL, about 0.15 mg/mL, about 0.16 mg/mL, about 0.17 mg/mL, about 0.18 mg/mL, about 0.19 mg/mL, about 0.20 mg/mL, about 0.21 mg/mL, about 0.22 mg/mL, about 0.23 mg/mL, about 0.24 mg/mL, about 0.25 mg/mL, about 0.26 mg/mL, about 0.27 mg/mL, about 0.28 mg/mL, about 0.29 mg/mL, about 0.30 mg/mL, about 0.31 mg/mL, about 0.32 mg/mL, about 0.33 mg/mL, about 0.34 mg/mL, about 0.35 mg/mL, about 0.36 mg/mL, about 0.37 mg/mL, about 0.38 mg/mL, about 0.39 mg/mL, about 0.40 mg/mL, about 0.41 mg/mL, about 0.42 mg/mL, about 0.43 mg/mL, about 0.44 mg/mL, about 0.45 mg/mL, about 0.46 mg/mL, about 0.47 mg/mL, about 0.48 mg/mL, about 0.49 mg/mL, or about 0.50 mg/mL. In embodiments, the range may be any interval recited between the amounts recited herein.

### Reaction Vessels

In aspects of the present application, efficient large-scale methods are contemplated. Generally, in order to meet the demand of an efficient method, variables such as the reaction vessel or type of reaction process becomes more important. Described herein are two types of processes: (1) batch processes and (2) continuous processes each of which offers advantages and disadvantages.

A batch process refers to a process that involves a sequence of steps followed in a specific order. Batch processing involves the processing of bulk material in groups through each step of the process. Processing of subsequent batches must wait until the current is finished. While batch processing offers lower initial setup cost as an initial advantage, the overall cost of processing increases. Further, validated modeling studies confirm that the kinetics of in vitro transcription and co-transcriptional capping are equal for batch and continuous processing. In embodiments, the batch process reaction vessel can be a batch reactor.

Continuous process refers to the flow of a single unit of product between every step of the process without any break in time, substance or extent. With regard to in vitro transcription, especially as contemplated herein, continuous flow offers advantages such as space-time yield, increased speed and capacity leading to reduced lead times. In embodiments, the continuous process reaction vessel can be a continuous stirred tank reactor.

### Additives

In aspects of the present application, a number of additives are used herein. In embodiments, additives may include, but are not limited to, pyrophosphatase, RNase inhibitor, solvents, calcium chloride (CaCl₂), and dithiothriotol (DTT).

### Pyrophosphatases

As used herein “pyrophosphatases” also refers to diphosphatase. Pyrophosphatases are enzymes that are acid anhydride hydrolases that hydrolyze diphosphate bonds. Pyrophosphatases are used in in vitro transcription reactions for synthesizing large-scale RNA products as it prevents pyrophosphate from precipitating with magnesium ions, which thereby increases the rate of the in vitro transcription reaction. In embodiments, the pyrophosphatase is an inorganic pyrophosphatase.

### RNase Inhibitor

The present application addresses challenges in manufacturing compositions with mRNA known to one of skill the art, including the sensitivity and instability of the molecule. There are several factors that contribute to the instability and sensitivity: (1) the presence of RNases (e.g., 5′ exonucleases, 3′ exonucleases, and endonucleases), (2) RNA is more susceptible to electrophilic additions, alkylations, and oxidations, and (3) the increased rate of hydrolysis in a solution pH exceeding 6.

As used herein “RNase inhibitor” or “RI” refers to ribonuclease inhibitor. RNase inhibitors are large molecules, approximately 49 kDa in size and rich in both cysteine and leucine compared to typical proteins. The highly repetitive and rich leucine content allows a tight complex to form. Crystal structures of the RNase inhibitor and RNase A complex suggest that the interaction is largely electrostatic in nature for the protein-protein interactions. During in vitro transcription, RNase inhibitor protects the newly transcribed mRNA from nuclease attack.

In embodiments, the RNase inhibitor is added to the in vitro transcription mixture. In embodiments, the RNase inhibitor is added with another additive to the in vitro transcription mixture. In embodiments, the RNase inhibitor is added with inorganic pyrophosphatase. In embodiments, the RNase inhibitor is guanidium thiocyanate. In embodiments, the RNase inhibitor is guanidinium isothiocyanate. In embodiments, the RNase inhibitor can be used in an amount of about 0.10 μg/μL, about 0.11 μg/μL, about 0.12 μg/μL, about 0.13 μg/μL, about 0.14 μg/μL, about 0.15 μg/μL, about 0.16 μg/μL, about 0.17 μg/μL, about 0.18 μg/μL, about 0.19 μg/μL, about 0.20 μg/μL, about 0.21 μg/μL, about 0.22 μg/μL, about 0.23 μg/μL, about 0.24 μg/μL, about 0.25 μg/μL, about 0.26 μg/μL, about 0.27 μg/μL, about 0.28 μg/μL, about 0.29 μg/μL, or about 0.30 μg/μL.

### Solvents

In aspects of the present application, additives such as an organic solvent are contemplated.

In embodiments, the solvent is selected from a polar protic solvent. In embodiments, the polar protic solvent is selected from the group consisting of water, methanol, ethanol, and isopropanol. In embodiments, the solvent is selected from a polar aprotic solvent. In embodiments, the polar aprotic solvent is selected from acetonitrile. In embodiments, the solvent is selected from methanol (MeOH), ethanol (EtOH), isopropanol (i-PrOH), acetonitrile (CH₃CN or MeCN), and combinations thereof.

In embodiments, the concentration of ethanol used as a solvent in the reaction mixture of RNA transcription is from about 0.5% v/v to about 15% v/v. In embodiments, the concentration of ethanol used as a solvent in the reaction mixture of RNA transcription is about about 0.5% v/v, about 1% v/v, about 1.5% v/v, about 2% v/v, about 2.5% v/v, about 3% v/v, about 3.5% v/v, about 4% v/v, about 4.5% v/v, about 5% v/v, about 5.5% v/v, about 6% v/v, about 6.5% v/v, about 7% v/v, about 7.5% v/v, about 8% v/v, about 8.5% v/v, about 9% v/v, about 9.5% v/v, about 10% v/v, about 10.5% v/v, about 11% v/v, about 11.5% v/v, about 12% v/v, about 12.5% v/v, about 13% v/v, about 13.5% v/v, about 14% v/v, about 14.5% v/v, or about 15% v/v.

In embodiments, the concentration of methanol used as a solvent in the reaction mixture of RNA transcription is from about 0.5% v/v to about 15% v/v. In embodiments, the concentration of methanol used as a solvent in the reaction mixture of RNA transcription is about 0.5% v/v, about 1% v/v, about 1.5% v/v, about 2% v/v, about 2.5% v/v, about 3% v/v, about 3.5% v/v, about 4% v/v, about 4.5% v/v, about 5% v/v, about 5.5% v/v, about 6% v/v, about 6.5% v/v, about 7% v/v, about 7.5% v/v, about 8% v/v, about 8.5% v/v, about 9% v/v, about 9.5% v/v, about 10% v/v, about 10.5% v/v, about 11% v/v, about 11.5% v/v, about 12% v/v, about 12.5% v/v, about 13% v/v, about 13.5% v/v, about 14% v/v, about 14.5% v/v, about 15% v/v.

In embodiments, the concentration of isopropanol used as a solvent in the reaction mixture of RNA transcription is from about 0.5% v/v to about 15% v/v. In embodiments, the concentration of isopropanol used as a solvent in the reaction mixture of RNA transcription is about 0.5% v/v, about 1% v/v, about 1.5% v/v, about 2% v/v, about 2.5% v/v, about 3% v/v, about 3.5% v/v, about 4% v/v, about 4.5% v/v, about 5% v/v, about 5.5% v/v, about 6% v/v, about 6.5% v/v, about 7% v/v, about 7.5% v/v, about 8% v/v, about 8.5% v/v, about 9% v/v, about 9.5% v/v, about 10% v/v, about 10.5% v/v, about 11% v/v, about 11.5% v/v, about 12% v/v, about 12.5% v/v, about 13% v/v, about 13.5% v/v, about 14% v/v, about 14.5% v/v, or about 15% v/v.

In embodiments, the concentration of acetonitrile used as a solvent in the reaction mixture of RNA transcription is from about 0.5% v/v to about 10% v/v. In embodiments, the concentration of acetonitrile used as a solvent in the reaction mixture of RNA transcription is about 0.5% v/v, about 1% v/v, about 1.5% v/v, about 2% v/v, about 2.5% v/v, about 3% v/v, about 3.5% v/v, about 4% v/v, about 4.5% v/v, about 5% v/v, about 5.5% v/v, about 6% v/v, about 6.5% v/v, about 7% v/v, about 7.5% v/v, about 8% v/v, about 8.5% v/v, about 9% v/v, about 9.5% v/v, or about 10% v/v.

In some embodiments, the total concentration of the one or more solvents does not exceed 10% v/v of the transcription mixture.

### Role of CaCl₂

As used herein “DNase I” refers to an deoxyribonuclease I, which is an endonuclease that non-specifically cleaves single- and double-stranded DNA. It hydrolyzes phosphodiester bonds producing mono- and oligodeoxyribonucleotides with 5′-phosphate and 3′—OH groups. Calcium chloride, which provides Ca²⁺, is an additive that has been found to be required for DNase I activity with Mg²⁺. Without being bound to any one theory, it is suggested that Ca²⁺ ions play an important role in the structural integrity of DNase I, whereas other metal cations such as Mg²⁺ and Mn²⁺ bind to the DNA substrate itself (Pan, C. Q. and Lazarus, R. A. Protein Science 1999, 8, 1780-1788). In crystal structures of bovine DNase I, “there are two distinct Ca²⁺ binding sites that stabilize two surface loops as well an additional metal ion binding site at the active site.” (Pan and Lazarus, 1999 citing Oefner C. and Suck D. J Mol. Biol. 1986, 192, 605-632.) Accordingly, the presence of calcium chloride was found to play an integral part the DNAse digestion reaction that occurs after the in vitro transcription as contemplated herein.

### DTT

As used herein, “DTT” refers to dithiothreitol or Cleland's reagent, an additive contemplated herein for in vitro transcription. DTT is a reducing agent used to reduce disulfide bonds of proteins to prevent intramolecular and intermolecular disulfide bonds forming between the cysteine residues of proteins. In embodiments, DTT is used to prevent dimerization. In embodiments, DTT is used as an additive to improve separation of proteins during electrophoresis by denaturing proteins.

In some embodiments, the present disclosure is drawn to a composition for in vitro transcription of a nucleic acid, the composition comprising a) ribonucleotide triphosphates (rNTPs), wherein the rNTPs consist of adenine (A), cytosine (C), guanine (G), and uracil (U) at a molar ratio of 10:13:13:6 (A:C:G:U); b) Mg²⁺; c) a DNA template; and d) RNA polymerase.

In some embodiments, the present disclosure is drawn to a kit for in vitro transcription of a nucleic acid, the kit comprising a) ribonucleotide triphosphates (rNTPs), wherein the rNTPs consist of adenine (A), cytosine (C), guanine (G), and uracil (U) at a molar ratio of 10:13:13:6 (A:C:G:U); b) Mg²⁺; c) a DNA template; and d) RNA polymerase.

### EXAMPLES

The following examples are offered for purposes of illustration and are not intended to limit the scope of the claims provided herein. All literature citations in these examples and throughout this specification are incorporated herein by references for all legal purposes to be served thereby. The process of in vitro transcription as generally carried out in the disclosure of WO 2024/006978 is incorporated herein by reference, as is the entire disclosure in WO 2024/006978 in its entirety.

### Example 1—In Vitro Transcription

Template DNA was linearized using BspQI enzyme for one hour at 50° C. The linearized DNA was optionally spiked with NaCl to produce a final concentration of 62.3 or 82.3 mM NaCl in the IVT. The IVT was assembled by combining Tris-HCl pH 7.5, DTT, rNTPs, magnesium acetate, ethanol, pyrophosphatase, RNase inhibitor, linearized DNA, capping reagent, and T7 RNA polymerase. For the DOE experiment in Table 2, the IVTs were prepared using an automated liquid handler. IVTs were incubated at 37° C. in a heat block for 60 to 180 minutes. At the end of the 37° C. incubation, the DNA template was degraded using DNAse I enzyme for 15 minutes at 37° C. followed by quenching with EDTA pH 8.0.

RNA integrity was assessed by Agilent Fragment Analyzer. RNA was prepared using the Agilent RNA Kit and run according to the manufacturer's protocol. The RNA integrity was computed using Prosize analysis software by computing the velocity-corrected main RNA peak area divided by the total peak area using the smear analysis tool.

Yield of the IVT was assessed using standard silica column purification followed by Nanodrop determination of the eluted RNA concentration.

NTP and cap concentrations were determined using HPLC and a Bia CIMmultus PrimaS analytical chromatography column. EDTA-quenched IVT samples were diluted in water and transferred to HPLC vials. Samples were injected on to the PrimaS column using the method in Table 1. The column eluent UV absorbance was measured at 260 nm and 280 nm wavelengths. In addition to experimental samples, standards were also injected for RNA, NTPs, and Cap to assign peaks in experimental samples. Resulting chromatograms were analyzed as follows. First, a blank run of sample diluent (water) was subtracted from all chromatograms. Second, peaks were labeled according to the residence time of rNTP, cap, and RNA standards. Notably, CTP and UTP peaks overlap and must be deconvoluted using the peak ratio of A260 and A280.

TABLE 1
PrimaS HPLC Method

| | | | 1M Tris | | | |
|---|---|---|---|---|---|---|
| | Time | Flow | HCl pH | | 1M | |
| Phase | (min) | (mL/min) | 8.0 | 5M NaCl | NaOH | Water |
| Equilibration | −2.5 | 1.5 | 5% | 0% | 0% | 95% |
| Inject sample | 0 | 1.5 | 5% | 0% | 0% | 95% |
| Salt gradient | 1 | 1.5 | 5% | 0% | 0% | 95% |
| | 5 | 1.5 | 5% | 50% | 0% | 45% |
| NaOH Wash | 5.1 | 1.5 | 0% | 20% | 10% | 70% |
| | 9.9 | 1.5 | 0% | 20% | 10% | 70% |
| Equilibration | 10 | 1.5 | 5% | 0% | 0% | 95% |
| | 12.5 | 1.5 | 5% | 0% | 0% | 95% |

### Example 2—Improved Methods to Increase Yield and Decrease Degradation

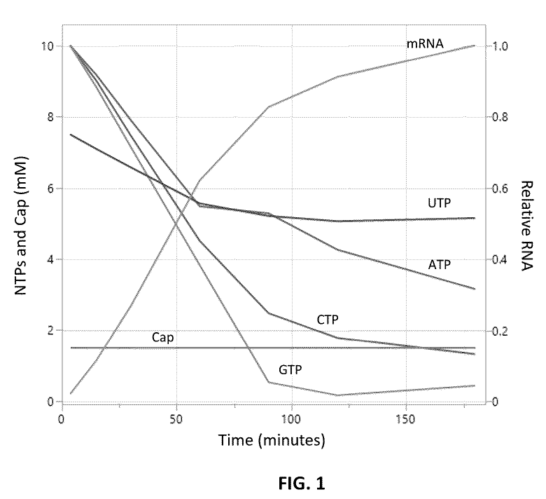

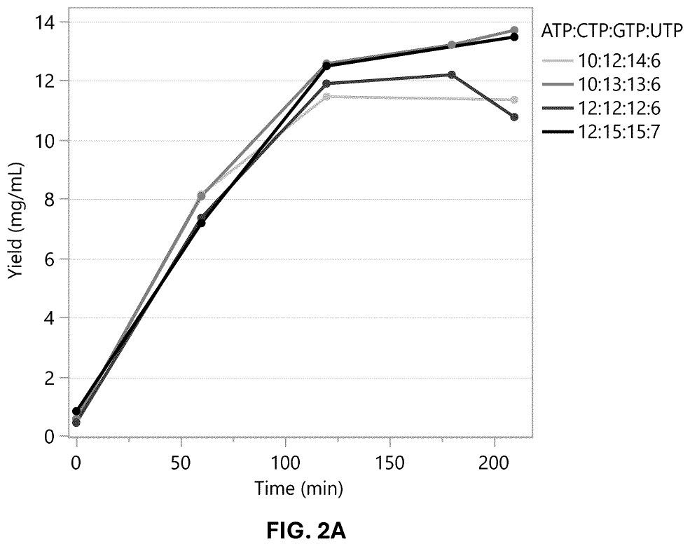

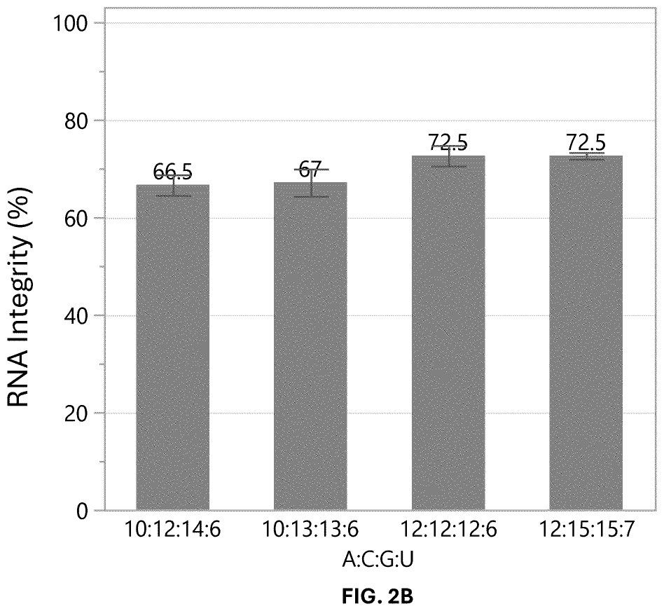

Certain NTPs appeared to be depleting faster than others (FIG. 1), a factor that limited the maximum mRNA yield of the IVT reaction. Based on this observation and nucleotide ratio in the sequence in this example (24% A, 31% C, 30% G, 14% U), the NTP ratios in the IVT reaction were varied and the effect of this variation on yield and mRNA quality was investigated (FIGS. 2A and 2B). The kinetics of the IVT reaction were then closely studied by manipulating other factors in the IVT reaction, such as the concentration of NaCl, the concentration of T7 RNA polymerase, the concentration of Mg²⁺, and reaction time (Table 2). For the DNA template used for this example, the adjusted NTP ratio of 10:13:13:6 and 12:15:15:7 (A:C:G:U) closely match the stoichiometric ratio of the template but vary in the total amount of NTPs added to the IVT (42 mM and 49 mM, respectively). The stoichiometric ratio of 12:15:15:7 (A:C:G:U), 125 U/μL T7 polymerase, and 62.3 mM NaCl gave the best balance of purity and yield. In this case, the improved IVT condition (samples 4 and 11) gives significantly higher yields with similar purity as the standard IVT condition (sample 1).

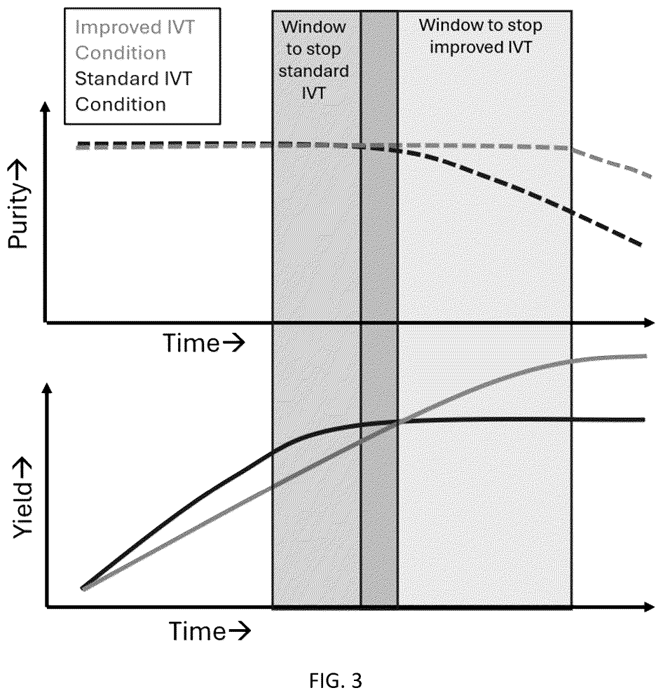

In other cases, it is apparent that this improved condition gives similar yield but higher purity (Table 3). This discrepancy can be explained by the kinetics of the reaction (FIG. 3). For any IVT reaction, the purity of the RNA will decrease at a faster rate when the NTPs are depleted because no more full-length RNA is being made to offset the degradation of the existing RNA. Because the kinetics of the improved condition will always be slower and the overall yield can be higher, there is a greater window to stop the reaction when both a high yield and purity are obtained (FIG. 3). This is important because the time at which the IVT will deplete the NTPs and reach maximum yield can vary slightly from reaction to reaction. In all cases, the purity of the improved condition will be as high or higher than the standard condition with the benefit that in some cases the yield will also be higher.

TABLE 2
IVT Reaction Output for Various Experimental Conditions.
The Conditions in sample numbers 4 and 11 showed
the best results. Condition 1 is the standard
IVT condition as a baseline comparison.

| | | | | | RNA | |
|---|---|---|---|---|---|---|
| | | Fold increase | | | Transcript | |
| | NaCl | in NTPs | T7 | A:C:G:U | Integrity | Yield |
| Sample | (mM) | Concentration | (U/μL) | Ratio | (%) | (g/L) |
| 1 | 82.3 | 1 | 125 | 12:12:12:6 | 81% | 11.5 |
| 2 | 62.3 | 1 | 250 | 10:13:13:6 | 77% | 12.9 |
| 3 | 62.3 | 1 | 250 | 12:12:12:6 | 77% | 12.4 |
| 4 | 62.3 | 1.15 | 125 | 10:13:13:6 | 81% | 18.0 |
| 5 | 82.3 | 1 | 125 | 12:12:12:6 | 76% | 12.7 |
| 6 | 82.3 | 1.15 | 250 | 10:13:13:6 | 72% | 16.9 |
| 7 | 82.3 | 1 | 125 | 10:13:13:6 | 77% | 14.0 |
| 8 | 82.3 | 1.15 | 250 | 12:12:12:6 | 73% | 15.9 |
| 9 | 82.3 | 1 | 125 | 10:13:13:6 | 78% | 12.3 |
| 10 | 82.3 | 1.15 | 250 | 10:13:13:6 | 72% | 15.3 |
| 11 | 62.3 | 1.15 | 125 | 10:13:13:6 | 82% | 15.6 |
| 12 | 62.3 | 1.15 | 125 | 12:12:12:6 | 82% | 14.0 |
| 13 | 62.3 | 1 | 250 | 12:12:12:6 | 80% | 12.3 |
| 14 | 62.3 | 1 | 250 | 10:13:13:6 | 80% | 13.2 |
| 15 | 82.3 | 1.15 | 250 | 12:12:12:6 | 77% | 15.0 |
| 16 | 62.3 | 1.15 | 125 | 12:12:12:6 | 22% | 0.0 |

TABLE 3
Comparison of improved condition to the standard condition.
The improved condition gives better purity for similar yield.

| | | RNA |
|---|---|---|
| | PrimaS Yield | Integrity |
| | (g/L) | (%) |
| Sample | Mean | Mean |
| Standard IVT condition | 11.6 | 72.0% |
| stopped at 120 min | | |
| Standard IVT condition | 10.6 | 72.2% |
| stopped at 180 min | | |
| Improved IVT condition | 12 | 81.0% |
| stopped at 120 min | | |

### Example 3. Use of Chelators to Improve Purity of RNA

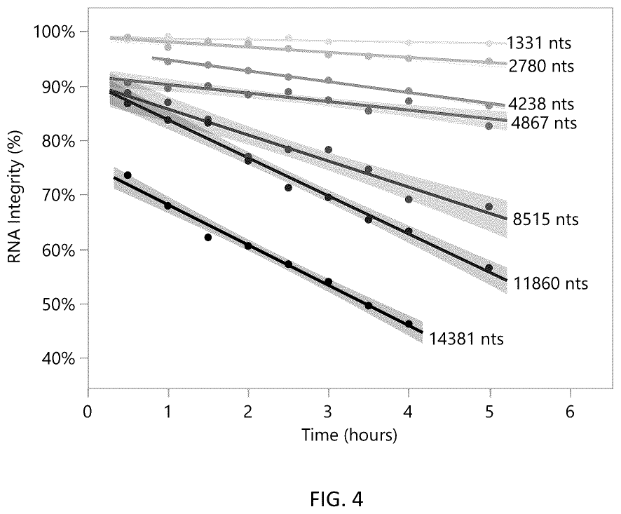

The integrity of RNA is a critical quality attribute of the final RNA product. RNAs that are not full length can be either inactive or produce incomplete proteins, both of which are undesirable. Furthermore, a primary determining factor for RNA product shelf life is the integrity of the RNA. The lower the integrity of the final product, the shorter the shelf life. This is especially a concern for long RNAs such as self-amplifying RNAs which are generally larger than 7,000 nts in length and as large as 15,000 nts in length due to the inclusion of replicase coding sequence. The degradation of RNA integrity is directly related to the RNA size. The integrity of long RNAs decreases more rapidly than with short RNAs (see FIG. 4). For RNAs under approximately 4000 nts, the degradation rate is nearly negligible compared to RNAs above 4,000 nts (FIG. 4). Therefore, maximizing RNA integrity for long RNAs is critical.

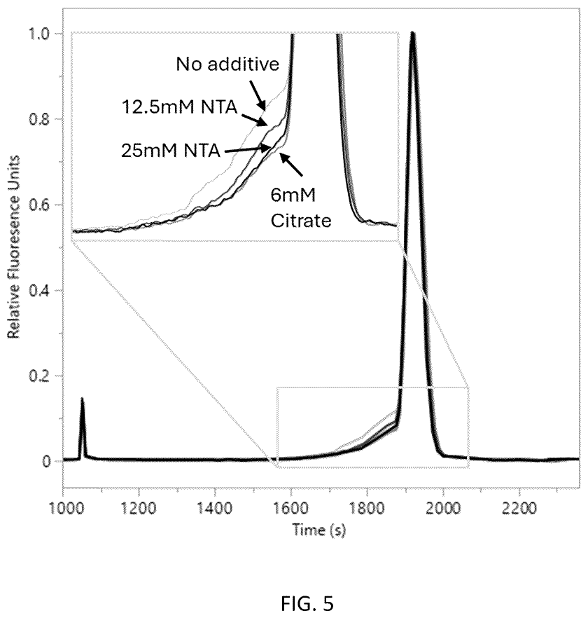

In accordance with these observations, the goal was to improve the integrity (purity or % full-length) of long RNAs. Including chelators in the IVT reaction improved RNA integrity of a self-amplifying RNA (>10,000 nts) by 5-8%, from 78% to ˜85% (Table 4 and FIG. 5). However, chelators negatively impacted the yield of the IVT, depending on concentration.

TABLE 4
Addition of chelators improve RNA integrity
after the IVT but reduce the final RNA yield.

| | | Additive | Yield | RNA |
|---|---|---|---|---|
| Sample | Additive | Concentration (mM) | (g/L) | Integrity (%) |
| 1 | None | 0 | 9.9 | 78% |
| 2 | EGTA | 50 | 3.7 | 86% |
| 3 | NTA | 50 | 3.1 | 87% |
| 4 | NTA | 25 | 8.4 | 86% |
| 5 | NTA | 12.5 | 10.1 | 83% |
| 6 | Citrate | 25 | 0.7 | Not Tested |
| 7 | Citrate | 12.5 | 1.8 | 82% |
| 8 | Citrate | 6 | 8.6 | 85% |

It was hypothesized that the yield decrease was partly due to the addition of excess sodium when adjusting the pH of the chelators with sodium hydroxide to near neutral pH. To test this hypothesis, the pH of NTA was adjusted with either Tris base or ammonium hydroxide. Furthermore, the sodium chloride concentration was reduced, and the concentration of T7 RNA polymerase was increased two-fold. The NTA that was adjusted with ammonium hydroxide (samples 4 and 5 in Table 5) produced crude IVT samples that had both high purity and good yield (sample 4: 89% at 9.6 g/L) compared to sodium hydroxide (samples 2 and 3) and tris (samples 6 and 7).

TABLE 5
NTA chelator adjusted to neutral pH with either sodium hydroxide
(NaOH), ammonium hydroxide (NH₄OH), or Tris base show different
effects on yield and RNA integrity, demonstrating that certain salts
can negatively impact RNA integrity and yield. NH₄OH shows best
combination of integrity and yield (sample 4).

| | | Additive | Yield | % RNA |
|---|---|---|---|---|
| Sample | Additive | Concentration (mM) | (g/L) | Integrity |
| 1 | None (control) | — | 12.9 | 77% |
| 2 | NTA NaOH | 50 | 9.9 | 81% |
| 3 | NTA NaOH | 25 | 12.4 | 81% |
| 4 | NTA NH₄OH | 50 | 9.6 | 89% |
| 5 | NTA NH₄OH | 25 | 12.9 | 85% |
| 6 | NTA Tris | 50 | 4.9 | 88% |
| 7 | NTA Tris | 25 | 13.1 | 86% |

Additional studies are focused on testing other chelators, in particular non-carboxylic acid chelators, e.g., phosphonic acid chelators, which chelate magnesium with phosphate groups instead of carboxylic acid groups. An example of phosphonic acid chelators are nucleotide triphosphates. A summary of the tested chelators is shown in Table 6.

TABLE 6
Qualitative summary of tested chelators

| Chelator | Result |
|---|---|
| L Glutamine | Lower RNA integrity |
| Citrate | High RNA integrity, higher dsRNA |
| EDTA | High RNA integrity, higher dsRNA |
| EGTA | High RNA integrity |
| NTA | Highest RNA integrity, low dsRNA, |
| | good yield |
| Ammonium salicylate | Slightly higher RNA integrity |
| Acetyl acetone | No change |
| Glycine | Lower RNA integrity |
| Quadrol | No change |
| EDTPO (Ethylene diamine tetra | High RNA integrity, higher dsRNA |
| methylene phosphonic acid) | |
| NTPO (Nitrilo tri methyl | No change |
| phosphonic acid) | |
| HDPO (Etidronic acid) | High RNA integrity, higher dsRNA |

Overall, nitrilotriacetic acid (NTA) was the best performer of chelators tested. While other chelators may improve RNA integrity, they can also negatively impact other RNA quality attributes. For example, the addition of ethylenediamine tetraacetic acid (EDTA), citrate, and EDTPO increase dsRNA. The best performing alternative to NTA is ethylenediamine tetra(methylene phosphoric acid) (EDTPO). EDTPO has the same structure as EDTA but has phosphonic acid groups instead of carboxylic acid groups. While RNA integrity improvement with EDTPO is similar to NTA (Table 7, sample 1 versus samples 5 and 8), EDTPO increases dsRNA in a dose-dependent manner (Table 7). In contrast, NTA reduces dsRNA in dose-dependent manner (Table 7).

TABLE 7
EDTPO chelator versus NTA chelator

| | | | | | RNA | | |
|---|---|---|---|---|---|---|---|
| | T7 | NaCl | NTA | EDTPO | Integrity | Yield | dsRNA |
| Sample | (U/μL) | (mM) | (mM) | (mM) | (%) | (g/L) | (ppm) |
| 1 | 125 | 62.3 | 0 | 0 | 73% | 9.4 | 16.6 |
| 2 | 250 | 0 | 0 | 0 | 65% | 10.2 | 48.2 |
| 3 | 250 | 0 | 12.5 | 0 | 74% | 10.4 | 35.2 |
| 4 | 250 | 0 | 25 | 0 | 79% | 10.4 | 17.5 |
| 5 | 250 | 0 | 50 | 0 | 83% | 9.5 | 10.0 |
| 6 | 250 | 0 | 0 | 5 | 75% | 9.8 | 37.2 |
| 7 | 250 | 0 | 0 | 7.5 | 80% | 9.7 | 39.8 |
| 8 | 250 | 0 | 0 | 10 | 83% | 9.7 | 51.4 |

Another area that was investigated was whether the free magnesium can be reduced by adding EDTA or by simply reducing the magnesium concentration in the IVT. Reducing the magnesium acetate (MgOAc) added to the IVT reaction (Table 8, samples 8-12) improves integrity ˜9% compared to the control (Table 8, sample 1), but not as much as NTA (13% increase; Table 8, sample 2). Furthermore, reducing magnesium significantly increases dsRNA. EDTA improves integrity, but also increases dsRNA (Table 8). The mechanism of how different chelators affect dsRNA is unknown.

TABLE 8
Compare addition of NTA to addition of EDTA or simply lowering
the Magnesium acetate (MgOAc) added to the IVT.

| | | | | | | RNA | | |
|---|---|---|---|---|---|---|---|---|
| | | | | | | Integ- | | |
| Sam- | MgOAc | NaCl | NTA | EDTA | T7 | rity | Yield | dsRNA |
| ple | (mM) | (mM) | (mM) | (mM) | (U/μL) | (%) | (g/L) | (ppm) |
| 1 | 50 | 62.3 | 0 | 0 | 125 | 67% | 12.4 | 105 |
| 2 | 50 | 0 | 50.0 | 0.0 | 250 | 80% | 10.9 | 103 |
| 3 | 50 | 0 | 0.0 | 10.0 | 250 | 71% | 12.1 | 124 |
| 4 | 50 | 0 | 0.0 | 15.0 | 250 | 80% | 5.0 | 210 |
| 5 | 50 | 0 | 0.0 | 20.0 | 250 | N/A¹ | 0.6 | N/A¹ |
| 6 | 50 | 0 | 0.0 | 25.0 | 250 | N/A¹ | 0.1 | N/A¹ |
| 7 | 50 | 0 | 0.0 | 30.0 | 250 | N/A¹ | 0.1 | N/A¹ |
| 8 | 50 | 0 | 0.0 | 0.0 | 250 | 57% | 12.2 | 141 |
| 9 | 45 | 0 | 0.0 | 0.0 | 250 | 67% | 14.5 | 173 |
| 10 | 40 | 0 | 0.0 | 0.0 | 250 | 76% | 12.1 | 230 |
| 11 | 35 | 0 | 0.0 | 0.0 | 250 | 65% | 1.8 | N/A¹ |
| 12 | 30 | 0 | 0.0 | 0.0 | 250 | N/A¹ | 0.3 | N/A¹ |

¹Yield too low for evaluation of RNA integrity and dsRNA levels

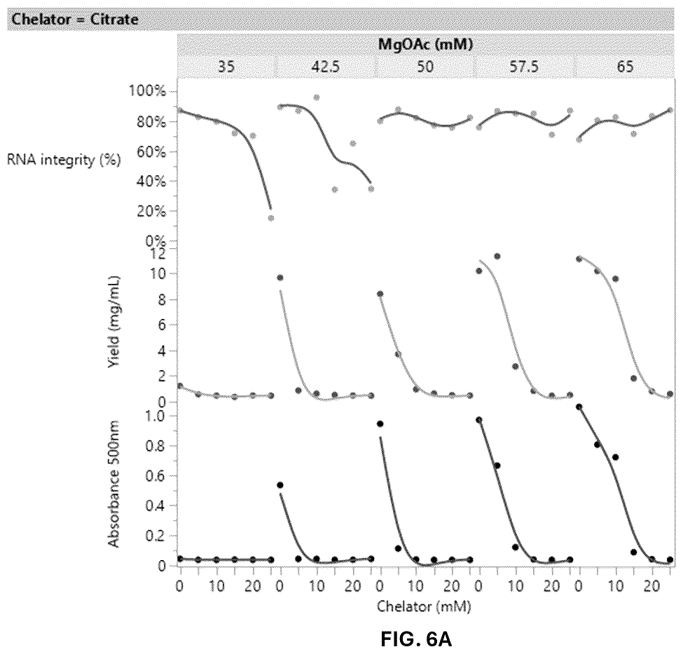

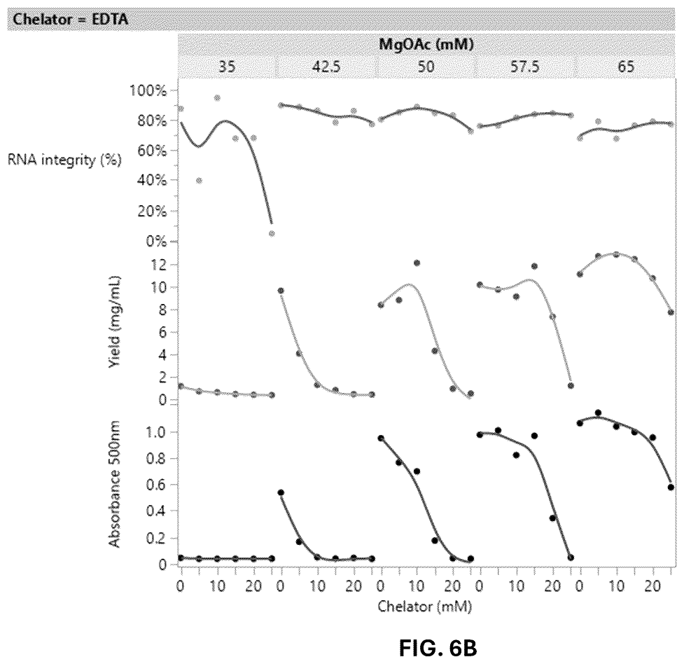

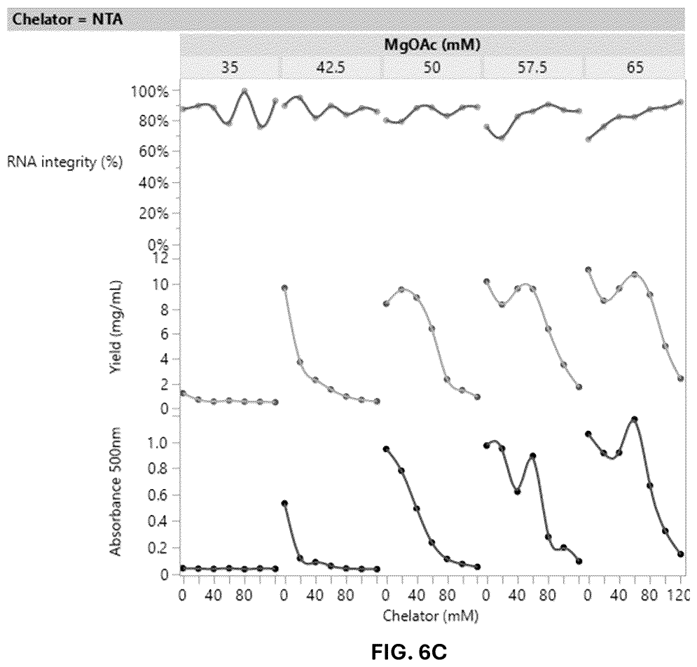

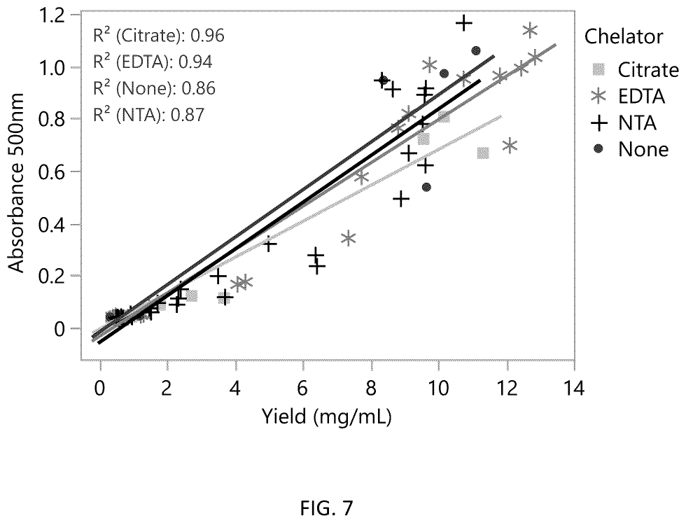

To better compare the operating region of various chelators, the amount of chelator and the amount of magnesium in an IVT reaction were varied using three different chelators. The yield, RNA integrity, and turbidity of the reaction were measured. The yield of the IVT drops off very sharply for citrate (FIG. 6A) and EDTA (FIG. 6B) above 15 mM of chelator, whereas NTA produces high yields up to 80 mM (FIG. 6C). This suggests that these weak chelators can be categorized into two or more groups with different mechanism/performance. Furthermore, these data show that the turbidity of the reaction is directly related to yield and negligibly affected by whether chelator is present or not (FIG. 7).

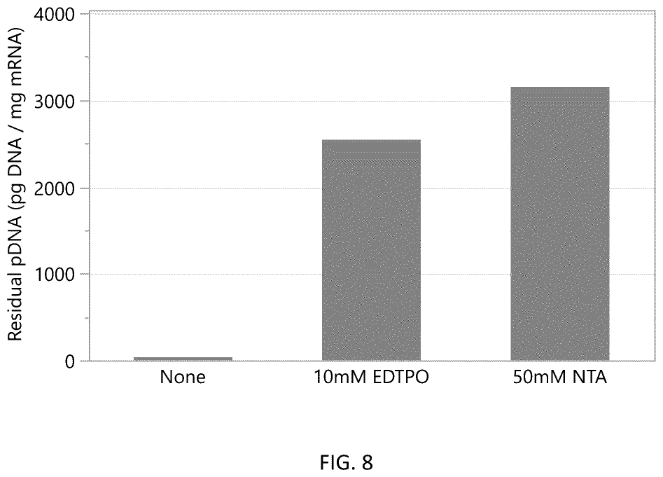

The effect of these weak chelators on the downstream DNase I reaction that digests the linearized DNA template was also investigated. After the IVT, DNAse I enzyme in a buffer containing calcium and magnesium ions was added. In the presence of NTA or EDTPO in the IVT, the residual plasmid DNA (pDNA) remaining after the DNAse reaction was significantly increased (FIG. 8). Without being bound by theory, it is presumed that the divalent cations required by DNAse I enzyme are at least partially chelated.

An unexpected finding was that there appear to be two concentration ranges where the tested chelators are most effective. This is unexpected as all the tested chelators have sufficiently high affinity for magnesium that it would be expected that the amount of chelated magnesium would be nearly equal to the concentration of chelator added (i.e. 10 mM chelator would chelate 10 mM magnesium). EDTPO, EDTA, and citrate all perform most effectively near 10 mM, which is near the expected amount of free magnesium in the IVT (42 mM total NTPs also chelate magnesium, leaving −8 mM free). In contrast, NTA and EGTA are most effective near the concentration of total magnesium (50 mM). This further emphasizes that it is not trivial to find the chelator and chelator concentration that improves integrity while maintaining or further improving other RNA quality attributes. Without being bound by theory, it is hypothesized that magnesium chelated by NTA and EGTA is still available for the T7 enzyme to use, whereas T7 is unable to utilize the magnesium chelated by the chelators most effective near 10 mM concentration. There is precedent for this effect for other enzymes. For example, DNAse I enzyme is still active with citrate-chelated manganese, but not EDTA-chelated manganese.

The examples and embodiments described herein are for illustrative purposes only and in some embodiments, various modifications or changes are to be included within the purview of disclosure and scope of the appended claims.

1-107. (canceled)

108. A method for producing a single-stranded RNA product, the method comprising:
a) reacting a transcription reaction mixture comprising a buffer solution comprising Mg²⁺, a DNA template, ribonucleoside tri-phosphates (rNTPs), and RNA polymerase; wherein the rNTPs comprise adenine (A), cytosine (C), guanine (G), and uracil (U) nucleosides, or analogs thereof, at a molar ratio that is within 5-10% of the ratio of A:C:G:T in the DNA template; and
b) controlling the rate of the transcription reaction of step (a) to be about 10 mM rNTP/hour to about 25 mM rNTP/hour, thereby producing the single-stranded RNA product.

109. The method of claim, 108 further comprising:
c) stopping the transcription reaction by digesting the DNA template with deoxyribonuclease (DNase) or quenching the RNA polymerase with a chelator; thereby producing the single-stranded RNA product.

110. The method of claim 109, wherein the
(i) chelator is selected from NTA (nitrilotriacetic acid), EDTA (ethylenediaminetetraacetic acid), EDTPO (ethylenediamine tetra(methylene phosphoric acid)), EGTA (ethylene glycol-bisO-aminoethyl ether)-N,N,N′,N′-tetraacetic acid, BAPTA (1,2-Bis(2-aminophenoxy)ethane-N,N,N′,N′-tetraacetic acid), DFOA (Deferoxamine Mesylate), Dimethoxynitrophenamine (1-(2-Nitro-4,5-dimethoxyphenyl)-1,2-diaminoethane-N,N,N′,N′-tetraacetic Acid)), CDTA (1,2-cyclohexylenedinitrilo)tetraacetic acid), DPTA (diethylenetriaminepentaacetic acid), PIH (pyridoxal isonicotinoyl hydrazone), and TPEN (N′-Tetrakis(2-pyridylmethyl)ethylenediamine); or
(ii) the chelator comprises NTA at a concentration of about 50 mM.

111. The method of claim 108, wherein the DNA template is in a solution comprising 0 mM to 1200 mM NaCl to produce a salt-spiked DNA template prior to introduction to the transcription reaction mixture.

112. The method of claim 108, wherein the DNA template is in a solution comprising 50 mM to 1200 mM NaCl to produce a salt-spiked DNA template prior to introduction to the transcription reaction mixture.

113. The method of claim 108, wherein
(i) the rNTPs are in a concentration ranging from about 25 mM to about 55 mM; or
(ii) the RNA polymerase is in a concentration of about 50 units/μL to about 400 units/μL.

114. The method of claim 108, wherein
(i) the method produces about 14 to about 20 g/L of single-stranded RNA product; or
(ii) the method produces about 10 to about 20 g/L of single-stranded RNA product.

115. The method of claim 108, wherein the single-stranded RNA product is about 75% to about 100% pure.

116. The method of claim 109, wherein the transcription reaction is stopped after about 60 minutes to about 240 minutes.

117. The method of claim 108, wherein the DNA template is linearized.

118. The method of claim 117, wherein an RNA polymerase KU activity per mL of transcription reaction mixture is greater than or equal to 125 KU RNA polymerase activity.

119. The method of claim 108, wherein the reaction mixture of step (a) further comprises RNase inhibitor, inorganic pyrophosphatase, or both.

120. The method of claim 108, wherein
(i) the initial pH of the reaction mixture is in a range from about 6.5 to about 8.0; or
(ii) step (a) and step (b) are each independently conducted at a temperature in a range from about 30° C. to about 40° C.; or
(iii) the RNA polymerase is selected from T7 polymerase, Escherichia coli (E. coli) polymerase, SP6 polymerase, T3 polymerase, or mutants thereof.

121. The method of claim 108, wherein the transcription reaction mixture of step (a) further comprises
(i) an initiating RNA capping reagent; or
(ii) one or more solvents selected from the group consisting of ethanol in a concentration of about 1 to about 10% v/v, isopropyl alcohol in a concentration of about 1 to about 10% v/v, methanol in a concentration of about 1 to about 10% v/v, and acetonitrile in a concentration of about 1 to about 8% v/v.

122. The method of claim 108, wherein
(i) the rate of reaction is controlled by having the initial total rNTP concentration be in the range of about 20 mM to about 60 mM; or
(ii) the rate of reaction is controlled by having the initial total rNTP concentration be in the range of about 20 mM to about 60 mM and the Mg²⁺ concentration be in the range of about 15 mM to about 80 mM.

123. The method of claim 117, wherein the ratio of adenine nucleosides, guanine nucleosides, cytosine nucleosides, and uracil nucleosides, or analogs thereof, are tuned to be within about 5% of the ratio of adenine, guanine, cytosine, and thymine bases on the linear DNA template.

124. The method of claim 123, wherein a yield of RNA transcript is increased by at least 5%, at least 10%, at least 15%, at least 20%, or at least 25% as compared to a transcription reaction in which the ratio of adenine nucleosides, guanine nucleosides, cytosine nucleosides, and uracil nucleosides, or analogs thereof, has not been tuned to be within about 5% of the ratio of adenine, guanine, cytosine, and thymine bases on the linear DNA template.

125. The method of claim 108, wherein the rNTPs comprise adenine (A), cytosine (C), guanine (G), and uracil (U) nucleosides, or analogs thereof, at a molar ratio of about 10:13:13:6 (A:C:G:U).

126. The method of claim 108, wherein
(i) the rate of the transcription reaction of step (a) is controlled to be about 13 mM rNTP/hour to about 20 mM rNTP/hour; or
(ii) the rate of transcription is controlled by increasing the concentration of rNTPs and/or decreasing the concentration of RNA polymerase; or
(iii) the rate of transcription is controlled by changing the temperature, salt concentration, magnesium concentration, pH, or a combination thereof.

127. The method of claim 108, wherein the RNA transcript is at least 4000 nucleotides in length.

128. The method of claim 110, wherein
(i) the chelator comprises NTA, and wherein the method further comprises adding ammonium hydroxide to the NTA prior to reacting the NTA with the transcription reaction mixture; or
(ii) the chelator comprises NTA, and wherein the method further comprises adding ammonium hydroxide to the NTA prior to reacting the NTA with the transcription reaction mixture, wherein the concentration of ammonium hydroxide is about 25 mM; or
(iii) further comprising adding additional chelator to the reaction as the reaction proceeds.

129. The method of claim 108, further comprising:
c) stopping the transcription reaction prior to a point in which the rNTPs have been depleted to between about 0% to 35% of the original amount of rNTPs in the reaction mixture; thereby producing the single-stranded RNA product.

130. A method for producing a single-stranded RNA product, the method comprising:
a) reacting a transcription reaction mixture comprising a buffer solution comprising Mg²⁺, a DNA template, ribonucleoside tri-phosphates (rNTPs), and RNA polymerase; wherein the rNTPs comprise adenine (A), cytosine (C), guanine (G), and uracil (U) nucleosides, or analogs thereof, at a molar ratio that is within 5-10% of the ratio of A:C:G:T in the DNA template, and wherein the transcription reaction results in the depletion of rNTPS, and
b) stopping the transcription reaction prior to a point in which the rNTPs have been depleted to between about 0% to 35% of the original amount of rNTPs in the reaction mixture; thereby producing the single-stranded RNA product.

131. The method of claim 130, wherein the purity of the RNA transcript product as determined by measuring the percentage of full-length RNA in the RNA transcript product is increased as compared to an in vitro transcription wherein the transcription reaction was not stopped when the rNTPs have been depleted to about 0% to about 35% of the original amount of rNTPs in the reaction mixture.

132. The method of claim 130, wherein the reaction is stopped by digesting the DNA template with deoxyribonuclease (DNase) or quenching the RNA polymerase with a chelator; thereby producing the single-stranded RNA product.

133. The method of claim 130, further comprising controlling the rate of the transcription reaction of step (a) to be about 10 mM rNTP/hour to about 25 mM rNTP/hour.

134. A method for reducing the degradation of an RNA transcript having a size larger than 4000 nucleotides, comprising reacting a transcription reaction mixture comprising a buffer solution comprising Mg²⁺, a DNA template, ribonucleoside tri-phosphates (rNTPs), and RNA polymerase; wherein the rNTPs comprise adenine (A), cytosine (C), guanine (G), and uracil (U) nucleosides, or analogs thereof; with a chelator.

135. The method of claim 134, wherein
(i) the chelator comprises EDTPO or EDTA at a concentration ranging from about 5 mM to about 50 mM and/or NTA or EGTA at a concentration ranging from about 1 mM to about 120 mM; or
(ii) the chelator comprises NTA, and wherein the method further comprises adding ammonium hydroxide, sodium hydroxide, or Tris base to the NTA prior to reacting the NTA with the transcription reaction mixture; or
(iii) the chelator comprises NTA, and wherein the method comprises adding ammonium hydroxide at a concentration ranging from about 20 mM to about 60 mM and/or adding sodium hydroxide at a concentration ranging from about 20 mM to about 60 mM; or
(iv) the chelator comprises NTA, and wherein the method comprises adding Tris base at a concentration ranging from about 20 mM to about 60 mM; or.
(v) the chelator comprises a non-carboxylic acid chelator.

136. A composition for in vitro transcription of a nucleic acid, the composition comprising:
a) ribonucleotide triphosphates (rNTPs), wherein the rNTPs consist of adenine (A), cytosine (C), guanine (G), and uracil (U) at a molar ratio of about 10:13:13:6 (A:C:G:U);
b) Mg²⁺;
c) a DNA template; and
d) RNA polymerase.

137. A kit for in vitro transcription of a nucleic acid, the kit comprising the components of the composition of claim 136.
